(19) United States
(12) Patent Application Publication
(10) Pub. No.: US 2025/0355005 A1
(43) Pub. Date: Nov. 20, 2025

(54) System and Method for Drug-Related Data Collection and Analysis

(71) Applicant: FloBio LLC, Philadelphia, PA (US)

(72) Inventors: Mark Allen Kunkel, Lawrenceville, NJ (US); Jason Matthew Rossi, Philadelphia, PA (US); Gary Lee Feiss, Telford, PA (US)

(73) Assignee: FloBio LLC, Philadelphia, PA (US)

(21) Appl. No.: 19/288,663

(22) Filed: Aug. 1, 2025

Related U.S. Application Data
(63) Continuation of application No. PCT/US2024/014159, filed on Feb. 2, 2024.
(60) Provisional application No. 63/442,847, filed on Feb. 2, 2023.

(51) Int. Cl. G01N 33/86; B01L 3/00

(52) U.S. Cl. CPC G01N 33/86; B01L 3/502715; B01L 2200/16; B01L 2300/0654; B01L 2300/0816; B01L 2300/0877; G01N 2500/02; G01N 2500/20

(57) ABSTRACT

An example system for detecting and quantifying drug and/or chemical interactions with a biological sample is provided. The system includes a detection instrument with computing capability. The system includes an assay device capable of receiving a biological sample. Introduction of the biological sample into the assay device results in a biological process by which fibrin and platelets may accumulate at a reaction zone of the assay device. The assay device is capable of receiving one or more chemical reagents and one or more drug reagents. The fibrin and platelets, and their associated signals, accumulated at the reaction zone of the assay device are usable to determine at least one of a drug presence, a drug class, a drug level in relation to a threshold, or a drug concentration, within the biological sample.

### CROSS-REFERENCE TO RELATED APPLICATIONS

This application is a continuation application that claims priority benefit to International Patent Application No. PCT/US2024/014159, filed on Feb. 2, 2024, which claims priority to U.S. Provisional Patent Application Ser. No. 63/442,847, filed on Feb. 2, 2023, the disclosures of which are incorporated herein by reference in their entireties.

### STATEMENT OF GOVERNMENT SUPPORT

This invention was made with government support under Award No. R44HL149480-03 awarded by the Department of Health and Human Services of the National Institutes of Health (NIH). The government has certain rights in the invention.

### BACKGROUND

Assessment of bleeding and clotting risk in emergency critical care environments may be difficult with traditional systems. Traditional systems are generally unable to create a testing environment that is analogous to the blood clotting physiology found in the human body, do not provide specific data on the key components of hemostasis (platelets and coagulation via fibrin formation), and cannot provide results in a timeframe required for critical care decision making in emergent situations. For example, FIGS. 1A-1C illustrate the physiology of hemostasis within the human body, with hemostasis involving a physiological response to vessel damage to arrest blood leakage involving both platelet aggregation and coagulation, with coagulation involving a system of enzymatic reactions that generate thrombin and fibrin that stabilize the clot. FIG. 1A illustrates vasoconstriction with collagen and tissue factor exposed upon vessel injury. FIG. 1B illustrates a platelet plug formation with platelet aggregation. FIG. 1C illustrates clot formation in the form of coagulation.

### SUMMARY

### Definitions

Specific terminology is defined here to clarify specific elements of the disclosure:

The term “system” refers to the entirety of the technology in question, including the microfluidic device, reagents, imaging instrument, software, analytical methods, and reporting.

The term “device” refers to the microfluidic apparatus for producing fibrin and platelet signals.

The term “reagents” refers to the chemicals and drugs necessary to perform the assay.

The term “instrument” or “analyzer” refers to the imaging apparatus and associated computing hardware used to collect and process data from the device.

The term “analytics” refers to the methodology for converting the raw imaging data into clinical results.

The term “reporting” refers to the documentation of the analytical results.

As used herein, the term “Factor IIa” is the scientific term for Thrombin. The conventional nomenclature for coagulation factors uses Roman numerals, Inhibitors to these factors are denoted by a small I, therefore the inhibitor to Factor Xa is named Xai, while the inhibitor to Factor IIa is IIai. As used herein, the term “DT” is a generic reference to Factor IIa, and can refer to the function of the DOAC drug to its target (a Direct Thrombin inhibitor, as compared to a Xai which is an indirect thrombin inhibitor). Factor II (pro-thrombin) is activated to Factor IIa (thrombin) through a pathway dependent upon Factor Xa. The terms “Factor IIa” or “IIa” are interchangeably used herein to refer to DT (and vice versa), and the terms “Factor IIai” or “IIai” are interchangeably used herein to refer to DTi (and vice versa). FIGS. 46-47 are diagrammatic views of a coagulation pathway relating to the use of term DTi and IIai herein

As used herein, the term “fluidically” or “fluidic” refers to a communication that has static or active fluid communication between the ports and along the fluidic paths. Because the device is fluidically connected, flow within the device is possible at any time. As used herein, the term “active” flow or “fluid communication” refers to flow that is brought about by pressure or vacuum applied to the fluidics of the device.

### Unmodified vs Modified Sample

A number of processes, effects, chemicals, and drugs are used in the system described herein. An important distinction is what is meant by a Modified or Unmodified Sample. A whole blood sample taken directly from a patient (whether containing drugs or not), and that same blood sample mixed with detection chemicals, is considered to be unmodified (as the term is used herein) because the behavior of the sample is not substantively changed from that of the in vivo behavior. For example, the addition of a platelet label, or the addition of a fibrinogen label does not intrinsically change the behavior of platelets nor of coagulation (e.g. fibrin accumulation). A modified sample (as the term is used herein) is one in which the intrinsic behavior has been modified from the in vivo condition. For example, an anticoagulant or antiplatelet medication, and/or the reversal agent of those same drugs applied to the sample will assuredly change the behavior of the platelets and/or fibrin. This is distinct from the fact that the sample may already contain some or all of these drugs inherently by way of the patient taking their prescribed medications.

### Indirect vs Direct Drug Effects

An indirect effect from a drug is one in which the physiologic response to the drug can be measured at processes that are distinct and/or distal to the molecular target for which the drug was designed. A direct drug effect is one in which the physiologic response to the drug can be measured at the drugs intended target. For example, Direct Thrombin Inhibitors (DTi, or IIai) like dabigatran directly target the activity of thrombin, while Factor Xa Inhibitors (Xai) like apixaban target factor Xa, which is a key upstream activator of thrombin. For thrombin, dabigatran has a direct effect on thrombin activity, while apixaban has an indirect effect on thrombin activity, even though both reduce thrombin activity.

### Inhibition vs Attenuation

In the example above, thrombin inhibition by either class of DOAC reduces fibrin formation. Neither class of drug directly inhibits fibrin; therefore, the reduction is an attenuation of fibrin development. Dabigitran directly inhibits thrombin, while Xa inhibitors like apixaban inhibit Factor Xa; but both attenuate fibrin by reducing the activity of thrombin, (and indirect effect).

### Drugs vs Chemicals

The utility of the system described herein is dependent upon the distinction of a drug vs a chemical. A drug is a specific compound formulated to derive a targeted biological effect. For example, the DOAC drug Dabigatran specifically targets (inhibits) thrombin. A chemical in this context is a material that performs a specific targeted function within the assay, but is not designed or intended to direct or influence biological pathways or function. For example, a platelet labeling chemical is used to fluorescently tag platelets. But, this chemical does not affect the way in which platelets interact biologically in the formation of a clot.

### Level, Concentration and Threshold

In general, the utility of evaluating a patient's sample for drug presence can be delineated into two paths: semi-analytical and analytical. A more general approach to understanding the effects of a drug on hemostasis is to determine the Level of the drug in the patient, typically in relation to a Threshold. As used herein, the term “Threshold” refers to a specific target value, or range of values, that is clinically meaningful when considering whether the level or concentration of a drug (or the activity of platelets or fibrin) is above, below or equal to this specific value or within a range of values.

A Level is a semi-quantitative assessment, as it may not definitively determine the absolute amount present in the blood. For example, a clinician may want to know if a patient's drug level is above or below a given Threshold (e.g. >or <30 ng/mL) for determining whether to give a DOAC reversal agent. In this case, the value of the result is in determining if the patient's drug level is above or below a given threshold (or within a given range of possible concentrations (e.g. >30 ng/mL but <100 ng/ml), not what the exact amount is. In contrast, the determination of Concentration is quantitative in that the actual amount of drug, +/− some level of inaccuracy, in the blood sample is determined.

### Molar Excess vs Supra-Therapeutic

For the purposes of this document, a supra-therapeutic dose of drug refers to any drug concentration that is in excess of the typical range of concentrations achieved in the blood plasma for normal adherence to the drug's medication dosing regimen. A “molar excess” of drug refers to any dose for which the corresponding assay signal which is modified by the presence of the drug (platelet fluorescence or fibrin fluorescence or other) would not be further attenuated by a higher dose of the drug. In this way, a “molar excess” can be considered a “signal saturating” dose of the drug.

Embodiments of the present disclosure provide for general and specific systems wherein the state of a patient's hemostatic function can be determined by measuring platelet and fibrin accumulation simultaneously, with accumulation of platelets, and fibrin as the reporting signals. Because hemostasis involves both platelet function and coagulation, the system evaluates both together to have a clear and accurate sense of a person's state of hemostatic function. While platelets (a subcellular component of hemostasis) are distinct from coagulation (an enzymatic reaction cascade), the two are intimately related to one another. Platelet binding to collagen and subsequent activation is a preemptive step in initiating a stabilized coagulation cascade, with degranulating platelets releasing a number of compounds that facilitate and stimulate coagulation as well as additional platelet accumulation. This process is affected by many co-factors including exposed membrane phospholipids that support the assembly of tenase and prothrombinase complexes. The terminal coagulation protease, thrombin, cleaves fibrinogen to form fibrin monomers and begin the formation of fibrin polymer. Fibrin formation generates a polymer mesh around aggregating platelets to form a stabilized clot. In addition to catalyzing fibrin, thrombin is also a potent activator of platelets. Therefore, modulators of platelet function specifically (such as targeted drugs) can also have differential levels of impact on fibrin formation, and vice versa. The two are intimately intertwined.

In general, blood products given to patients can have an immediate effect on platelet and/or fibrin function. Packed red blood cells affect hematocrit, which directly affects platelet margination and therefore concentration in the cell free layer. Direct purified platelet addition immediately increases platelet function. Fresh frozen plasma contains all of the cofactors for coagulation and four-factor prothrombin concentrate (4-FPCC) contains key co-factors for coagulation that can be used both for bleeding events, and to reverse the effects of anti-coagulants like warfarin. (See, e.g., Horstman, E. E. et al., Plasma Products for Transfusion: An Overview, Department of Laboratory Medicine, Yale University School of Medicine, New Haven, CT, USA, Vol. 7 (March 2022)). Therefore, blood product use and stewardship directly requires the need to monitor both platelet and coagulation function.

Hemostatic equilibrium is a key aspect of normal hemostatic function where the body regulates both platelet function and coagulation so as not to create a physiologic response that is either too weak, nor too great, to injury or disease state. While in general there are “normal” measurable amounts of platelets, fibrinogen and other coagulation factors circulating in the blood, there are a multitude of additional factors that can influence any one individual's hemostatic response to injury or disease, thereby creating a wide range of “normal” function. This represents a major hurdle for diagnostic evaluation of hemostatic function that only evaluates one aspect of the hemostatic cascade.

Beyond tissue injury, many factors can disturb the normal functioning of platelets and the coagulation cascade. These include, e.g., drugs, illness, disease, genetics, certain foods, physical injuries, combinations thereof, or the like. Published research shows that, for example, physical trauma to the body can lead to a dysfunction of platelet activity. COVID-19 infection has been shown to lead to hyper coagulation, that can lead to inappropriate clot formation and death. Suppression or enhancement of platelet function and/or coagulation can lead to either severe bleeding events or hyper-clotting events, respectively, that can complicate medical care, lead to iatrogenic injury, and even lead to death. Certain medical conditions that require medication or procedures that modify platelet function and/or coagulation (such as anti-coagulant or anti-platelet drugs, pro-coagulant drugs, blood transfusions, packed red blood cells, platelet therapy, or the like) can also lead to abnormal clotting and/or bleeding events that can cause iatrogenic injury or even death, with rapid onset.

From a medical perspective, general clotting function (hemostasis) is distinct from the medical process of preventing abnormal clotting (thrombosis), that is not related to an injury. For example, direct-acting oral anticoagulants (DOACs) that directly suppress coagulation function are taken by patients to prevent venous thromboembolism (VTEs), exacerbated by long term medical conditions, such as cardiac abnormalities and arrhythmias like aFib. In some instances, prevention of a clot can be of paramount importance, to avoid a stroke or heart attack, for example. This is distinct from preventing a clot formed by a direct physical injury (a short term process), because many patients are placed on DOAC therapy long term (months or years), and therefore have disturbed clotting behavior over long periods of time. In emergency critical care environments then, the detailed and accurate determination of whether a patient is taking DOACs, and their state of anti-coagulation, would be essential to properly managing the patient's increased bleeding risk caused by the DOAC drug. In conventional emergency critical care environments, the only available information may be if the patient is prescribed a DOAC and possibly when their last dose was taken (if this information is available at all). In such circumstances, the accuracy related to DOAC usage and the actual state of anti-coagulation within the patient can be difficult, if not impossible, to obtain. The exemplary systems discussed herein provide an accurate and detailed determination of whether a patient is taking DOACs and their state of anti-coagulation. In some embodiments, the systems discussed herein can be used for a similar determination for novel oral anticoagulants (NOACs), target-specific oral anticoagulant (TSOACs), and/or novel platelet targeting drugs, and/or drugs targeting other upstream aspects of the hemostatic cascade.

In its simplest case, a patient's blood is passed over a reaction zone where platelet and coagulation activators have been placed; for example collagen and lipidated tissue factor (LTF). As whole blood flows over the reaction zone, platelets can become bound to the collagen by way of their collagen receptors, which activate platelets. LTF in combination with factor VIIa and other co-factors activates the coagulation cascade. This activation then recruits more platelets to the clotting zone, and activated thrombin catalyses the formation of fibrin from soluble fibrinogen. This reaction is self-sustaining and additional platelet and fibrin accumulation increase over time until the flow of blood is occluded. The reaction zone could include only one reactant (collagen), or more than collagen and LTF.

Blood from a healthy, uninjured person would produce a specific profile of platelet and fibrin accumulation over time. Individual variances between healthy, uninjured persons would produce a distribution of “normal” hemostatic function around an average overall population function (sec, e.g., FIGS. 43 and 44). This would provide for a boundary of conditions where signals outside of this “normal” distribution would constitute “abnormal” platelet or coagulation function. In some embodiments, the assay could distinguish normal from abnormal platelet and coagulation function by a percentage or percentiles (for example) from the “normal” average (however this would not identify where the abnormal behavior was coming from (e.g., drugs, injury, disease, or the like).

Determining drug presence: If there is a drug present that targets platelet or fibrin function, then the drug's presence may be determined by the system or assay through a functional test, using a reversal agent that is specific for that drug. A reversal agent could be a specific drug formulated to reverse the activity of the target drug. If the drug's reversal agent is added to a patient blood sample containing the drug, then an increase in platelet and/or fibrin signal would be expected. In the absence of the drug, the reversal agent would show no effect (positive or negative) to platelet function or coagulation.

Determining class of drugs: If more than one class of drug exists that targets the same function (platelet or coagulation), the class of the drug may be determined through a functional test of platelet function and/or coagulation. For example, if there are two classes of drugs (A and B) that reduce fibrin formation (coagulation), and a reversal agent to A is added to a patient blood sample containing class A drug, then an increase in platelet and/or fibrin signal would be expected for that reaction. More specifically, if a second reaction was run where class B reversal agent was added to a patient blood sample containing class A drug, then no increase in platelet and/or fibrin signal would be expected for that reaction (and vice versa). In combination, using two separate reactions with reversal agent A and B, the coincident data would accurately identify which class of drug the patient was using. In the absence of the drug, neither reversal agent would show an effect (positive or negative) to platelet function or coagulation. The system or assay discussed herein can rely on a similar concept to identify one or more class of drugs existing in the patient blood sample.

IC50 Curve for the determination of drug concentration: In some embodiments, the system or assay discussed herein can determine an unknown drug concentration by comparing the amount of inhibition found in the patient's sample being evaluated to a known standard curve, generated from a healthy donor population analyzed by the device/system. In such embodiments, a series of healthy blood samples is treated with a drug, at different concentrations, that reduces platelet or fibrin function. A series of drug dilutions is made over the expected clinically functional range of drug. This standard curve of drug activity can then be used by the system or assay to compare to the patient's sample being evaluated. The amount of inhibition seen in the patient to be evaluated, is compared to the inhibition found in the standard curve. This percent inhibition is then correlated to the drug concentration that demonstrated the same level of inhibition in the healthy population. FIG. 41 provides an example of an IC50 curve which could be used in the described manner to determine drug concentration.

Method for determining clinical cutoffs for specific drugs: In some embodiments, the system can determine a useful clinical value by discriminating whether a drug concentration is above or below a specific threshold. This is important as there are clinical guidance documents that promote the use of reversal drugs in the event that a patient is above certain thresholds for coagulation inhibiting drugs. Statistical methods like Binary Logistic Regression, or Classification and Regression Tree (CART) discriminant analysis can be used by the system. In such embodiments, a set of donor specimens (spiked or patient specimens) can be evaluated by a true analytical approach, such as liquid chromatography/mass spectrometry (LCMS) analysis, to determine the actual concentration for the drug as the analyte. The drug concentration is then categorized on a binary basis relative to the threshold concentration (at or below, versus above). Using Binary Logistic Regression with the device's fibrin and platelet signals under the differing conditions, a regression equation is derived with which to classify samples with unknown amounts of drug present relative to the threshold concentration. In a similar approach, CART Classification utilizes the various fibrin and platelet signals from the device to determine or generate an algorithm which is used to classify unknown amounts of drug present relative to the threshold concentration. This approach allows for the use of fibrin and platelet (and potentially other) signals to be analyzed simultaneously to improve the concordance between LCMS and the test device, and hence the ability to determine an unknown.

Statistical method for determining drug concentration: In some embodiments, the system is capable of more precisely determining drug concentration. Statistical methods like Regression (e.g., linear regression), or Classification and Regression Tree (CART) regression analysis can be used. In such embodiments, a set of donor specimens (spiked or patient specimens) can be evaluated by a true analytical approach, such as liquid chromatography/mass spectrometry (LCMS) analysis, to determine the actual concentration for the drug as the analyte. Using Regression with the device fibrin and platelet signals under the different conditions, a regression equation is derived by the system with which to provide an estimate of the actual drug concentration. In a similar approach, CART Regression utilizes the various fibrin and platelet signals from the device to determine or generate an algorithm which is used to calculate estimated concentrations of drug in the sample. This approach allows for the use of fibrin and platelet (and potentially other) signals to be analyzed simultaneously to improve the concordance between LCMS and the test device, and hence the ability to determine an unknown.

In some embodiments, it may be advantageous to utilize regression equations for each specific drug that may be run on the system. For example, FIG. 50A shows the result of regression analysis for each drug based upon its own unique clinical and LC-MS/MS data. This data uses the log10 fibrin signals, unmodified and fully reversed as continuous variables and the drug identity (apixaban (A), dabigatran (D), or rivaroxaban (R) as categorical variables with the log10 concentration. Each equation is based on data for each of the three specific drugs. In comparison, if only drug class is used as a categorical input to predict the concentration as log10, the results are only two equations (i.e., two drug classes), with poor correlation demonstrated by the Xa regression equation to the drugs apixaban and rivaroxaban. (See FIG. 50B). When analyzing this same data (unknown drug but known drug class) using CART regression, the correlation is obvious (similar to the individual drug equations). (See FIG. 50C).

With the specific drug identity unknown and the DOAC class determined by the exemplary system/device, the average precision is 22.8% using the Regression method to determine the concentration, and 12.0% using the CART Regression method. When the specific drug is known, the average precision is 9.9%. This ability to select for precision is useful for several reasons. In cases where the clinician knows the correct drug the patient is taking, it would be possible to select for the individual drug regression model that is the best fit for that drug, thereby resulting in higher accuracy in determining drug concentration in the patient. This selectability of the regression model can most simply be done through the GUI interface and related system software. In the case where the drug is unknown to the clinician, the CART regression method can produce quantitative or semi-quantitative drug concentration results. This shows that the CART model in particular moderates the individual Xa drug behavior such that in CART, drug type has much less effect than it does in conventional Regression. In all cases, drug class is determined by the DOAC test's reversal agents.

Methods for performing the evaluation of platelet function and coagulation can be performed by the system in several ways, but should utilize blood that is under flow. Traditional systems often utilize constrained blood samples with minimal or controlled agitation (such as stirring) to measure clotting function. However, these methods are not able to distinguish the individual activity of coagulation from platelet function, may utilize fractionated blood products (such as plasma) that are only a proxy to a whole blood sample, and do not mimic the effective behavior of blood in vivo. The very act of fractionating blood into its constituent components also creates an artificial environment that is unlike that found in the body. Therefore, the activity of platelets and the coagulation pathway in this scenario is neither representative of in vivo blood behavior, nor of biochemical distribution of reactants found in the body. Other traditional systems that attempt to analyze blood under flow generally make no attempt to reproduce the actual blood flow characteristics of the body, resulting in an assay that does not represent the activity of blood in the body. Traditional devices that attempt to recapitulate blood flow often do not attempt to assess both platelet function and coagulation concurrently, providing an incomplete answer to overall hemostatic function. The ability then to accurately determine platelet and coagulation function concurrently, under physiologic conditions, is absolutely critical to evaluating hemostatic function, in real-time. The systems discussed herein provide such determination.

Several in vivo key characteristics determine blood behavior as it relates to platelet function and coagulation: (i) shear rate at the surface of the reaction zone; (ii) viscosity; (iii) temperature; (iv) flow/volume of blood; (v) surface area of the injury site; (vi) hematocrit; (vii) platelet count and function; (viii) fibrinogen levels; and/or (ix) coagulation co-factor levels. Platelets, for example, have over 10 different classes of receptors on their surface that play multiple roles in platelet function. (See, e.g., Saboor, M. et al., Platelet receptors; an instrumental of platelet physiology, Pak. J. Med. Sci., 29(3): 891-896 (May-June 2013)). The number of receptors, the distribution of the classes, and the overall function of the receptors, all play a role in overall platelet function that is affected by blood flow and reactant delivery to the platelet at the site of binding. Coagulation is driven by a multitude of cofactors, many of which are delivered to (and removed from) the site of coagulation by way of blood flow (and subsequent diffusion into and through the clot). These coagulation factors can also be affected by genetic variability and overall concentration in the blood (as many are produced in the liver), and are mediated by many types of drugs, food, and hormones, to name a few. Therefore, the interaction of platelets and coagulation factors at the site of clotting is dynamic and directly affected by diffusion which is impacted by blood flow, and distribution/concentration of blood components (such as fibrinogen).

Hemodynamic properties of platelets also affect hemostasis: Platelets in particular are affected by blood flow dynamics. Platelets are approximately 1-3 microns in size in comparison to biconcave red blood cells (RBC) which are roughly 7-8 microns in diameter. Due to the dynamics of RBCs, platelet function is influenced by a hemodynamic process where RBCs exclude platelets from the center of flowing blood, that marginates the platelets to the vessel walls. (See, e.g., Sugihara-Seki, M. et al., Margination of Platelet-Sized Particles in the Red Blood Cell Suspension Flow through Square Microchannels, Micromachines, 12, 1175 (2021) (https://doi.org/10.3390/mi12101175). This specifically increases platelet interaction with vessel walls (or device microfluidic channel surfaces). Therefore, a device that can also provide basic blood measures of hematocrit and platelet count (as well as other measures) would be greatly beneficial to understanding the performance of the assay in regard to platelet and fibrin signals. The system discussed herein could provide such measures by way of spectroscopic analysis of the blood in the fluidic paths, using published methods for determining hematocrit, oxy/deoxyhemoglobin and even platelet counts. (See, e.g., Lipowsky, H. et al., Hematocrit determination in small bore tubes by differential spectrophotometry, Microvascular Research, Vol. 24 (1), p. 42-55 (1982); Mattley, Y. et al., Light scattering and absorption model for the quantitative interpretation of human blood platelet spectral data, Photochem. Photobiol., Vol. 71 (5), p. 610-619 (2000); Kitamura, Y. et al., Spectrophotometric determination of platelet counts in platelet-rich plasma, International Journal of Implant Dentistry, Vol. 4 (29) (2018)).

Devices and methods to derive platelet and fibrin signals: In general, blood should be flowed over a reaction zone at initial wall shear rates that are physiologic. This ranges widely based upon vessel diameter and blood pressure, but in general ranges from 100 to 500 seconds⁻¹ for venous blood flow to 500 and much higher s⁻¹ for arterial blood flows. With stronger wall shear forces, different biological parameters are involved. For example, platelet binding in high arterial shear is directly impacted by von Willebrand Factor (vWF) that has little to no effects at venous shear. Since vWF is a key component of hemostasis, and known diseases affect vWF function, then an assay's sensitivity toward such factors can be “tuned” by changing the assays operating shear rate. While vessel diameters range widely in size (from 1 cm for large arteries to microns for capillaries), there are practical limits to what can be achieved from an assay perspective in terms of creating a physiologically representative model of hemostasis. Too small and the assay easily occludes from particulate debris, is difficult to manufacture, and/or generates significant backpressure. Too large and the amount of blood needed for an assay is impractical. For a typical blood draw, 1-3 mL of blood is common and does not inconvenience the patient in any way. This translates into a flow device that has small vasculature-like paths, on the order of tens to a few hundred microns in cross-section.

While vasculature in the body is cylindrical, platelet and fibrin behavior for blood flow through high aspect ratio rectangular paths approximates plane Poiseuille flow through infinite parallel plates. With flow paths of this size, 500 uL of blood can provide up to 15 minutes or more of clot formation time, which is more than sufficient to analyze platelet and fibrin behavior. The clot zone of the device can be a multitude of sizes, from only a few microns wide to millimeters in length. From a practical standpoint, a clotting zone of, e.g., 100-500 microns inclusive, 100-450 microns inclusive, 100-400 microns inclusive, 100-350 microns inclusive, 100-300 microns inclusive, 100-250 microns inclusive, 100-200 microns inclusive, 100-150 microns inclusive, 150-500 microns inclusive, 200-500 microns inclusive, 250-500 microns inclusive, 300-500 microns inclusive, 350-500 microns inclusive, 400-500 microns inclusive, 450-500 microns inclusive, 100 microns, 150 microns, 200 microns, 250 microns, 300 microns, 350 microns, 400 microns, 450 microns, 500 microns, or the like, allows for sufficient signal to accumulate over a 15 minute assay period. The clotting zone could be applied to all sides of the flow path (top, bottom, left, right), but can generate fully occlusive clots when applied to only one side of a geometric flow path (e.g. rectangular). Therefore, in some embodiments, the clotting zone can be applied to, e.g., the top side of the flow path, the bottom side of the flow path, the left side of the flow path, the right side of the flow path, combinations thereof, or the like. While rectangular flow paths are practical to produce, alternative flow path cross sectional shapes (e.g. semi-circular) are also contemplated. The clotting zone could include one reaction site, or could include multiple reaction sites within the same flow, and in proximity to one another. Detection of the accumulation of platelets and fibrin can be accomplished optically (e.g. direct fluorescence), or with any number of additional methods available to anyone skilled in the art.

In some embodiments, a device could include modification of the surfaces of the flow paths/fluidics (e.g., biologicals, proteins, silanes, chemicals, combinations thereof, or the like) in order to passivate their surfaces to avoid activation or inadvertent reactions with the blood sample. In other embodiments, surface modification of the flow paths/fluidics could include modification of the surface to more mimic the in vivo characteristics of the body (e.g., coating the surfaces with a fatty acid to mimic cellular membranes, or to literally grow endothelial cells within the device to form pseudo tissue like flow paths).

Utilizing a device to determine if a drug is present in the blood that could affect platelet function or coagulation is possible using a limited number of independent reactions. In addition, the concentration of a drug that affects platelet or fibrin function can also be determined by a limited number of independent reactions. In preferred embodiments, even the class of a drug that affects platelet or fibrin function can be determined using a limited number of independent reactions.

Platelet and Fibrin detection: In order to determine the state of platelet and fibrin function, a platelet label and fibrin label are applied to the blood sample being processed. This permits the simultaneous detection of platelets and fibrin at the same clotting zone of the device. This of course does not prevent one from using either label independently if desired, but in order to evaluate both fibrin function and platelet function together, both labels are applied to the same sample, at the same time. Only a brief incubation is required to label platelets, and no incubation time is required for fibrinogen. A platelet label for example could be an antibody to human CD61 (a platelet integrin) that is conjugated with Alexa 488. A fibrin label, for example, could be human fibrinogen conjugated with Alex 594. The fibrinogen-594 is added to the blood sample as a small proportion of the existing native fibrinogen, which is then incorporated into the forming clot as a proportion of total fibrin. Alternative detection methods are anticipated that utilize the basic functional biochemistry of clot formation. α2-antiplasmin (A2-APF) is a protein that impedes fibrinolysis, and is naturally cross-linked to fibrin during clot formation. (See, e.g., Liu, Y et al., Fluorescent peptide for detecting factor XIIIa activity and fibrin in whole blood clots forming under flow, Res. Pract. Thromb. Hacmost., 7;8(1): 102291 (December 2023), doi: 10.1016/j.rpth.2023.102291, PMID: 38222077, PMCID: PMC10787300). Fluorescently labeled A2-APF will incorporate into the growing clot coincident with fibrin, providing an additional means to monitor fibrin formation.

In some instances, plasma, instead of whole blood, may be useful to analyze. While plasma lacks the cellular components of whole blood, additions to the plasma, such as viscosity enhancers, freeze dried platelets, and other blood components could be added to create a material that could be analyzed by the device. In some instances, the blood (or plasma) sample may be pre-treated with chemicals to avoid complications of platelet function and coagulation, caused by the collection and handling of blood, such as contact activation. For example, Corn Trypsin Inhibitor (CTI) is a small protein that is localized in the kernels of most species of corn. CTI is not only an inhibitor of trypsin, but is also a specific human factor XIIa inhibitor. The inhibitor forms a one-to-one complex with either trypsin or factor XIIa, and when added to blood or plasma, prolongs the activated partial thromboplastin time without affecting the PT assay. The specificity for factor XIla makes the inhibitor useful for the segregation and study of tissue factor (TF) dependent coagulation reactions. The use of CTI to study TF-dependent reactions has been documented in literature. (See, e.g., Rand, M. D. et al., Blood clotting in minimally altered whole blood, Blood, Vol. 88 (9), p. 3432-3445 (1996); Cawthern, K M. et al., Blood coagulation in hemophilia A and hemophilia C, Blood, Vol 91 (12), p. 4581-4592 (1998); Dargaud, Y. et al., Platelet-dependent thrombography: a method for diagnostic laboratories, British Journal of Hematology, Vol. 134 (3), p. 323-325 (2006); Mann, K. G. et al., Citrate anticoagulation and the dynamics of thrombin generation, Journal of Thrombosis and Hemostasis, Vol. 5 (10), p. 2055-2061 (2007)).

Studies indicate that suppression of the contact pathway of coagulation is essential when attempting to perform TF-dependent assays in whole blood or plasma samples. The addition of CTI at the point of sample collection prevents activation of the contact pathway during subsequent sample processing steps, thus reducing in-vitro artifacts. The most common use of CTI is associated with thrombin generation assays when attempting to work at low TF concentrations. Additional chemicals can be used for the purpose of modifying blood and plasma behavior, prior to, and during use within a device. A common example of such a chemical is chloromethylketone (FPRCK (Phe-Pro-Arg-chloromethylketone; commonly referred to as PPACK), which is a rapid thrombin inhibitor and EGRCK (Glu-Gly-Arg-chloromethylketone; commonly referred to as GGACK), which is a rapid factor Xa inhibitor. Both FPRCK and EGRCK are used extensively during protein isolation procedures to inhibit serine protease activity and prevent further conversion of zymogens to active enzymes.

FIG. 42 shows various envisioned configurations of the exemplary device flow paths. It should be understood that the flow paths illustrated in FIG. 42 can be used in independent/separate devices, or multiple flow paths can be incorporated into a single device with any combination of single or multiple clot sites with the same or differing TF concentrations. Single or combined devices can utilize any combination of clot size and TF concentrations. In some embodiments, the flow path can include a single reaction chamber, and single independent flow path. (see FIG. 42, flow paths 1-3). For example, flow path 1 in FIG. 42 includes one independent flow path with one small clot site, and one TF concentration. Flow path 2 of FIG. 42 includes two independent flow paths with one large clot site, and one TF concentration. Flow path 3 of FIG. 42 includes three independent flow paths with three small clot sites, and one TF concentration.). In some embodiments, the flow path can utilize more than one concentration of TF. For example, flow path 4 of FIG. 42 includes one independent flow paths with clot sites having different TF concentrations located serially.

In other embodiments, a flow path can have more than one flow path, with independent reactions. (See FIG. 42, flow paths 5-6) Flow path 5 of FIG. 42 includes two separate paths and two different TF concentrations in parallel. Flow path 6 of FIG. 42 includes two separate paths with two reaction zones on separate planes (z-axis), with two different non-overlapping (z-axis) sites having different TF concentrations in parallel. Each flow path represented in FIG. 42 includes a reaction chamber fluidically connected to a reaction zone via a flow path, the reaction zone having one or more clot sites. The clot sites can have the same TF concentration or different TF concentrations. A flow path leads from the reaction zone to waste. Flow path 5 of FIG. 42 includes two separate, parallel flow paths with clot sites in an overlapping position, with the reaction zone on the same plane (along the z-axis). Flow path 6 of FIG. 42 includes two separate, parallel flow paths with clot sites in a non-overlapping configuration, with the reaction zone on different planes (along the z-axis).

In some embodiments, the independent flow paths can be connected to a common sample entry point to simplify the addition of a blood sample. In some embodiments, this common entry point can also include a common reagent chamber to facilitate the interaction of the blood sample with reagents common to all reactions (e.g., CTI, fibrin label, platelet label, combinations thereof, or the like). In some embodiments, each independent flow path can contain an independent reagent and/or mixing chamber where reagents unique to each flow path can be added, stored and/or mixed with the blood sample. In some embodiments, the flow paths can coincidently be connected to a priming circuit whereby pressure applied to the priming circuit can push fluid into the flow paths of the device, providing blocking of surfaces and elimination of air (which avoids bubble entrapment). A blood sample to be tested can first be mixed with CTI, then labeled with platelet label and fluorescent fibrinogen in a sample chamber, and mixed with specific drugs or reagents in the same or additional reagent/mixing chambers. This mixture can then be drawn through the device (under vacuum or pressure) and across the reaction zone. A single clotting site can provide both a platelet signal and a fibrin signal.

More than one reaction zone or clotting site can be included in the device to provide averaging of the platelet and fibrin signals. (see, e.g., FIG. 42). The clotting site can be of different sizes to facilitate more or less reactive surface area. A single flow path, for example, can be split into two or more independent clotting sites with independent or convergent exits. In some embodiments, a device can include independent flow paths and clotting sites on different layers of the device, creating a multi-dimensional flow path (in the x, y and z planes).

FIG. 51 shows additional diagrammatic representation of exemplary microfluidic device configurations. Pressure can be applied at the inlet to push blood through the device, vacuum can be applied at the exit to pull the blood through the device, or a combination of pressure and vacuum can be applied to move/oscillate blood back and forth through the device. The device can include a blood entry chamber (in some embodiments including CTI) that acts as an inlet for blood. The blood entry chamber is fluidically connected by flow paths to a sample chamber. Each device or flow path includes a reagent and mixing zone disposed upstream of a reaction zone having a clot site. One or more clot sites can exist in the reaction zone. Downstream of the reaction zone, the device/system includes a prime pump configured to apply a positive pressure to the flow path. Each flow path can be independent and leads to waste downstream, which optionally can be under a negative pressure (vacuum). As an example, the device of FIG. 51 can have the patient's neat blood, the patient's blood with Andexxa, the patient's blood with Praxbind, and the patient's blood with excess rivaroxaban, with reagents located in the reagents and the mixing zone.

FIGS. 52 and 53 show additional diagrammatic representations of exemplary microfluidic device pathway configurations. In particular, FIG. 52 is a diagrammatic view of a microfluidic device pathway including a multi-layer structure with two unique reaction zones, and FIG. 53 is a diagrammatic view of microfluidic device pathway including a single-layer structure with two unique reaction zones. Although it is technically possible to have two reaction zones within the same pathway, there may be technical difficulties this can create (e.g., upstream vs. downstream reactive zones conflicting one another). The flow paths of FIGS. 52 and 53 can provide parallel paths in three or two dimensions to allow for multiple reaction zones within the same device. For example, the device of FIG. 52 includes a layer 300 with a flow path 302 and a reaction zone 304, and a vertically offset layer 306 with a flow path 308 and a reaction zone 310. The three-dimensional configuration allows for two reaction zones offset in the z-axis direction on separate planes. As a further example, the device of FIG. 53 includes a single layer 320 with flow paths 322, 324 spaced in a two-dimensional manner, each flow path 322, 324 including its respective reaction zone 326, 328.

Multi-Level Drug Sensitivity: While fluidic devices can be made with a single reaction zone with a specific level of reactivity to platelet function and coagulation, it may be advantageous to have devices with more than one reaction zone with each reaction zone at a different level of reactivity. For example, Tissue Factor (TF) is the primary activator of the process of coagulation whereby a given amount of TF, bound within the reaction zone, will produce a specific coagulation response for any given sample. Collagen concentration can equally be modified to differing levels to modify the behavior of platelet binding and activation. This means that creating a second reaction zone with a differing level of TF and/or collagen will naturally produce a different level of reactivity to a given sample. This is useful, for example, when attempting to detect a drug which inhibits one or more reactions in the coagulation cascade. Different levels of tissue factor in the reaction zone will cause different rates of generation of the molecular targets of certain anticoagulants, which can then modulate the apparent activity of a given dose of drug. In the case of DOACs, two TF concentrations could be chosen such that one TF concentration in the reaction zone would be sensitive to a low level of DOAC drug, (e.g. <100 ng/ml or <50 ng/mL) while a second TF concentration in a second reaction zone would be more sensitive to a moderate to high dose of DOAC drug, (>100 ng/ml or >200 ng/mL. This is useful, for example, at resolving limitations in assay linearity or sensitivity where reaction kinetics at a given TF concentration may simply not result in measurable differences between similar drug levels. By having two distinct reaction zones with differing TF concentration, linearity or sensitivity can be maintained across a wide range of biologically relevant drug concentrations.

Multiple reaction zones within the same device can be utilized to provide varying degrees of reactivity to different biological scenarios, such as differing drug levels. For example, these different reaction zones can be placed sequentially within the same blood flow, in parallel within the same plane, where the same blood sample is split between two separate paths each crossing a unique reaction zone, or even dimensionally separated where one flow path/reaction zone is placed upon a separate fluidic layer in comparison to the first, all within the same device. (See FIG. 42).

In order to determine the presence of a single drug, utilizing a reversal agent, a device would need at least two independent reaction chambers (where blood and reagents are mixed together), two independent reaction zones, and two independent clotting sites. One independent flow path with independent clotting zone contains the blood sample with platelet and fibrinogen label. A second independent flow path with an independent clotting zone contains the blood sample with platelet and fibrinogen label, as well as the reversal agent for the drug in question. FIG. 42 provides a diagrammatic view of such a microfluidic device for determining the presence of a single drug using a reversal agent. The device includes reaction chambers that are fluidly connected to respective reaction zones, and that fluidly lead to waste. The first reaction zone would identify the state of platelet and coagulation function. The second reaction zone would identify the response of the blood sample to the reversal drug, by way of the fibrin and platelet signal at the second clotting site. In all cases, both flow paths produce both fibrin and platelet signals.

In order to determine the concentration of a drug, the device would include at least three independent flow paths. One independent flow path with a clotting zone would include the blood sample with platelet and fibrinogen label. A second flow path with a clotting zone would include the blood sample with platelet and fibrinogen label, as well as the reversal agent for the drug in question. A third flow path with a clotting zone would include the blood sample with platelet and fibrinogen label, as well as a supra-therapeutic dose of the drug in question that completely attenuates the fibrin signal. The first flow path provides a general assessment of the sample's platelet and fibrin function; the second flow path identifies the presence of the drug and provides for the maximum signal without the action of the inhibitor drug (either fibrin or platelets); the third flow path provides for the minimum signal with a saturating action of the inhibitor drug (fibrin or platelets). In general, it would be expected that either the platelet signal or the fibrin signal would be influenced most, as most drugs target either the coagulation pathway, or platelet function, not both. But in all cases, since the device provides both fibrin and platelet signals, both data points from each reaction zone can be used for analysis, and therefore a patient that has both types of drugs on board could be assessed.

In the case where drug class would need to also be determined, the device can include a minimum of four independent reaction zones and flow paths. One independent flow path with independent clotting zone would include the blood sample with platelet and fibrinogen label. A second independent flow path with independent clotting zone would include the blood sample with platelet and fibrinogen label, as well as the reversal agent for drug A in question. A third independent flow path with independent clotting zone would include the blood sample with platelet and fibrinogen label, as well as the reversal agent for drug B in question. A fourth independent flow path with independent clotting zone would include the blood sample with platelet and fibrinogen label, as well as a supra-therapeutic dose (molar excess) of one of the drugs in question. The results would be similar to those discussed above for a three independent flow path device, except that an additional data point would exist that would identify which class of drug was present by a comparison of the effects of the two reversal agents (where a response to one reversal agent identifies the drug class present). This methodology is broadly applicable to any drug that affects platelet function and/or coagulation and which has a reversal agent specific to that drug that can be used ex vivo.

### Direct Oral Anticoagulant (DOAC) Specific Test

In some embodiments, the methodology for the system or device can include the comparison of a patient's blood, to that same blood's interaction with the following: 1) patient's blood behavior in the presence of a reversal agent to the drug in question; 2) patient's blood behavior in the presence of a molar excess of the suspected drug. Behavior in this sense refers to the sample's characteristic platelet and coagulation response (although other responses are anticipated).

For example, a drug that affects coagulation would be expected to attenuate the patient's fibrin signal from “normal”. Applying that drugs reversal agent to a sample of the patient's blood prior to analysis would be expected to increase the patient's fibrin signal into the “normal” range, provided no other complications are present. This simple process where the patient's unmodified blood and the patient's blood treated ex vivo with the reversal agent and a molar excess of the same drug simultaneously would identify the presence and relative activity of the drug as described above, for each specific patient. That is to say, the patient's own blood is used as a control in the device to determine behavior to different drugs.

Detection of DOAC drugs as an exemplary diagnostic device: In particular, the method provides a means for detailed and accurate assessment of whether a patient is taking DOACs in order to decide whether clinical treatment to reverse DOAC activity is needed for proper treatment of the patient. The method provides rapid and more precise hemostasis testing (as compared to traditional systems), especially during the critical 10-20 minute triage window, and can be used to improve early bleeding risk assessment, stabilization, and care transition, while driving evidence based transfusions, blood product utilization, and costly DOAC reversal. The method provides results that can be used by medical professionals to reduce overall costs of patient care, while adhering to existing industry quality and patient safety guidelines.

In accordance with embodiments of the present disclosure, an exemplary system for direct-acting oral anticoagulant (DOAC) detection and quantification is provided. The system includes a first inlet port, a second inlet port, a third inlet port, and a fourth inlet port, each configured to receive a fluid sample. In some embodiments, the system can include more than four inlet ports, e.g., 8 inlet ports, or the like. The system includes an outlet port, and microfluidic flow paths fluidly connecting each of the first, second, third and fourth inlet ports with the outlet port. The unmodified sample can be labeled on or off device, before use. The labeling consists of a platelet specific label, and a fibrin specific label. Introduction of the labeled sample into the respective first, second, third and fourth inlet ports generates a fibrin and platelet signal. Additional reversal drugs are specifically and independently used to identify the class of DOAC drug present in the sample ((cither Xai or DTi), with Xai reversal drug in port 2, and DTi reversal drug in Port 3 for example) by reversing the effects of the DOAC agent and therefore producing a fully recovered fibrin signal. A molar excess of inhibitor (either Xai, DTi, or both) provides for a fully attenuated fibrin signal in port 4. Platelet signals in each port are expected to also be affected, even though the DOAC drugs are not targeting platelet function directly. This is because platelets are affected by the activity of thrombin, which is affected by the DOAC drugs, either directly (DTi) or indirectly (Xai). Therefore, there is an indirect effect on platelets by way of the coagulation pathway, in particular the formation of thrombin.

The fluid sample can be, e.g., a raw blood sample, a citrated blood sample that is recalcified, a heparin treated blood sample that is treated with protamine, or the like. When a DOAC is present in a patient's blood sample, the fibrin signal, the fully reversed fibrin signal (by class), and the corresponding coincident platelet signals in each port can be used to determine DOAC presence, and concentration in the sample. The fibrin signals, the fully reversed fibrin signal, the fully attenuated fibrin signal, and the coincident corresponding platelet signals can be used to determine a DOAC concentration or “level” in the fluid sample by multiple mathematical methods. In some embodiments, the first inlet port can be configured to receive the fluid sample in an unmodified manner, the second inlet port can be configured to receive the fluid sample and a Xai reversal agent, the third inlet port can be configured to receive the fluid sample and a DTi reversal agent, and the fourth inlet port can be configured to receive the fluid sample with a molar excess of a Xai inhibitor (fully suppressed signal). The drugs and samples can be combined either external to the device, or within the device.

The system can include a processing instrument configured to manipulate the sample within the device and coincidently monitor a direct response of the fluid sample to the reversal drugs to identify a DOAC class present in the fluid sample. In some embodiments, the DOAC class can be Factor Xai or Factor IIa (DTi). In some embodiments, the instrument can include a light source for monitoring clot development in the microfluidic flow paths. A green fluorescent signal can indicate platelet formation as the clot develops, a red fluorescent signal can indicate fibrin formation as the clot develops. The processing instrument can be configured to measure the fluorescent intensity of the monitored clots as they develop, and can be configured to correlate the measured fluorescent intensity with platelet and fibrin accumulation in the microfluidic flow paths. The processing instrument can be configured to compare the fibrin signal to the fully reversed/recovered fibrin signal, the fibrin signal to the fully attenuated fibrin signal, and to compare the coincident platelet signals for all reactions to determine a DOAC concentration level in the fluid sample.

In accordance with embodiments of the present disclosure, an exemplary method for direct-acting oral anticoagulant (DOAC) detection and quantification is provided. The method includes adding a fluid sample to a first inlet port, a second inlet port, a third inlet port, and a fourth inlet port of an assay device. The assay device includes an outlet port and microfluidic flow paths fluidly connecting each of the first, second, third and fourth inlet ports with the outlet port. In some embodiments, the assay device can include a separate priming port to pre-fill the device, or the priming could be accomplished by way of the outlet port. The method includes generating fibrin and platelet signal for each of the inlet ports. The method includes identifying the overall state of coagulation and platelet function from the unmodified sample (port 1), as well as class of DOAC drug present in the sample (either Xai or DTi) by evaluating fibrin signal using a Xai and IIai reversal agent in ports 2 and 3 respectively. A molar excess of Xa or IIa inhibitor provides for a fully attenuated fibrin signal in port 4. In all cases, a coincident platelet signal is also generated for each port.

It should be noted that a patient would typically never be prescribed both a Xai and DTi drug at the same time, and therefore only one reversal agent would be expected to produce a response if the patient were on a DOAC. However, the assay could produce a response even if both classes of drug were present. DOAC presence and class are detectable from the same data.

The method can include imparting a light source onto the microfluidic flow paths to monitor clot development in the microfluidic flow paths. The method can include the instrument receiving as input at a processing device a measured fluorescent intensity of the monitored clot development. The method can include correlating the measured fluorescent intensity with platelet and fibrin accumulation in the microfluidic flow paths, using the instrument. The method can include comparing with a processing instrument the fibrin signal to the fully reversed fibrin signal, the fully inhibited fibrin signal, and the coincident platelet signals. The fibrin and platelet signals confer information about the general state of platelet function and fibrin activity in the unmodified sample, as well as specific information about the sample's reactivity to Xai and DTi reversal agents, and Xa and/or DTi inhibitors. In some embodiments, platelet and fibrin function can be determined by comparing to population averages. DOAC class and concentration can be determined by comparing unmodified sample function to chemically or drug modified sample function.

In accordance with embodiments of the present disclosure, an exemplary system for direct-acting oral anticoagulant (DOAC) detection and quantification is provided. The system includes an optical instrument with computing capability, and an assay device capable of receiving a biological sample and further capable of receiving one or more chemical reagents. Introduction of the biological sample and the one or more chemical reagents into the assay device results in a biological process by which fibrin and platelets accumulate at a reaction zone of the assay device. The system includes a fluorescent assembly capable of detection of the biological process by way of fluorescent labeling and detection of a resulting accumulating fluorescent signal. The fibrin and platelets accumulated at the reaction zone of the assay device, and the accumulating fluorescent signal are usable to determine at least one of a DOAC presence, a DOAC class, a DOAC level in relation to a threshold, or a DOAC concentration, within the biological sample. In some embodiments, the system can be used for detection and quantification of any type of drug that affects platelet and fibrin behavior, and can thereby be used to determine at least one of a drug presence, a drug class, a drug level, or a drug concentration within the biological sample. In some embodiments, the system can used for detection and quantification of a DOAC specifically.

In some embodiments, the assay device can include a first inlet port, a second inlet port, a third inlet port, and a fourth inlet port, each configured to receive the biological sample. The biological sample is an unmodified sample including a platelet specific label and a fibrin specific label. The assay device can include an outlet port, and microfluidic flow paths fluidically connecting each of the first, second, third and fourth inlet ports with the outlet port. The introduction of the biological sample into the respective first, second, third and fourth inlet ports generates a fibrin signal and a platelet signal.

At least one of the first, second, third and fourth inlet ports can be configured to receive a reversal drug to identify a class of DOAC present in the biological sample. The reversal drug inhibits, antagonizes or attenuates the activity of a Xai or DTi DOAC. The reversal drug reverses the effects of a DOAC drug and produces a fully recovered fibrin signal. In some embodiments, at least one of the first, second, third, and fourth inlet ports can be configured to receive a concentration of either a Xai or DTi drug which completely inhibits the fibrin signal,, resulting in a fully attenuated fibrin signal.

In some embodiments, the system can include a processing device configured to receive as input the fibrin signal, the platelet signal, the fully recovered fibrin signal, and the fully recovered platelet signal, and determine a DOAC concentration or level in the biological sample. In some embodiments, the first inlet port can be configured to receive the biological sample in an unmodified manner, the second inlet port can be configured to receive the biological sample and a Xai reversal agent, the third inlet port can be configured to receive the biological sample and a DTi reversal agent, and the fourth inlet port can be configured to receive the biological sample with a molar excess of a Xa or DT inhibitor.

The biological sample can include a raw blood sample, a citrated blood sample, or a heparin treated blood sample. In some embodiments, the system can include a processing device configured to manipulate the biological sample and monitor a direct response of the biological sample to the one or more reversal drugs to identify a DOAC class present in the biological sample. In some embodiments, the DOAC class can be Factor Xai or Factor IIai (DTi).

In some embodiments, the system can include a light source for monitoring clot development in the microfluidic flow paths and detecting a fluorescent reaction of the one or more reagents. A green fluorescent signal can indicate platelet formation in the clot development. A red fluorescent signal can indicate fibrin formation in the clot development. In some embodiments, the system can include a processing device configured to receive as input a measured fluorescent intensity of the monitored clot development, and further configured to correlate the measured fluorescent intensity with platelet and fibrin accumulation in the microfluidic flow paths. In some embodiments, the system can include a processing device configured to compare the fibrin signal to the fully recovered fibrin signal, compare the fibrin signal to the fully attenuated fibrin signal, and compare coincident platelet signals for all reactions to determine a DOAC concentration or level in the fluid sample.

In accordance with embodiments of the present disclosure, an exemplary method for direct-acting oral anticoagulant (DOAC) detection and quantification is provided. In some embodiments, the method can be used for detection and quantification of any drug that affects clotting and that has a reversal agent (e.g., the drug presence, the drug class, the drug level in relation to a threshold, the drug concentration, or the like). The steps described herein can therefore be used in a non-DOAC specific manner. The method includes adding a biological sample to an assay device, adding one or more chemical reagents to the assay device to generate a biological process by which fibrin and platelets accumulate at a reaction zone of the assay device, detecting the biological process with a fluorescent assembly by way of fluorescent labeling and detection of a resulting accumulating fluorescent signal, and using the fibrin and platelets accumulated at the reaction zone of the assay device, and the accumulating fluorescent signal, to determine at least one of a drug presence, a drug class, a drug level in relation to a threshold, or a drug concentration.

The method can include adding the biological sample to a first inlet port, a second inlet port, a third inlet port, and a fourth inlet port of the assay device. The assay device can include an outlet port and microfluidic flow paths fluidly connecting each of the first, second, third and fourth inlet ports with the outlet port. The biological sample can be an unmodified sample including a platelet specific label and a fibrin specific label. The method can include generating a fibrin signal and a platelet signal from the biological sample for each of the first, second, third and fourth inlet ports. The method can include determining the drug class and the drug concentration from the fibrin signal and the platelet signal.

In some embodiments, the method can include identifying an overall state of coagulation from the unmodified sample by evaluating the fibrin signal and a fully recovered fibrin signal obtained using a reversal agent. In some embodiments, the method can include identifying a platelet function from the unmodified sample by evaluating the platelet signal and a fully recovered platelet signal obtained using a reversal agent. In some embodiments, the method can include using a Xai reversal agent in the second inlet port and a DTi reversal agent in the third inlet port. A molar excess of Xai or DTi DOAC in the fourth inlet port can provide a fully attenuated fibrin signal. For example, the method can include receiving a concentration of the drug to a point which produces no further attenuation of the fibrin signal to obtain the fully attenuated fibrin signal.

In some embodiments, the method can include generating a platelet signal coincident with the fibrin signal for each of the first, second, third and fourth inlet ports. In some embodiments, an unmodified sample fibrin and an unmodified sample platelet signal can be generated from a microfluidic flow path associated with the first inlet port, a fully reversed fibrin signal and a coincident platelet signal can be generated from either a microfluidic flow path associated with the second or third inlet port by way of interaction with a Xai or DTi reversal agent, and a fully attenuated fibrin signal and a coincident platelet signal can be generated from a microfluidic flow path associated with the fourth inlet port.

In some embodiments, the method can include imparting a light source onto the microfluidic flow paths to monitor clot development in the microfluidic flow paths based on a fluorescent reaction of the one or more reagents, receiving as input at a processing device a measured fluorescent intensity of the monitored clot development, and correlating with the processing device the measured fluorescent intensity with platelet and fibrin accumulation in the microfluidic flow paths. In some embodiments, the method can include comparing with a processing device the fibrin signal to a fully reversed fibrin signal, and a fully inhibited fibrin signal along with coincident platelet signals, to determine the DOAC concentration in the biological sample.

In accordance with embodiments of the present disclosure, an exemplary system for detecting and quantifying drug and/or chemical interactions with a biological sample is provided. The system includes a detection instrument with computing capability, and an assay device capable of receiving a biological sample. Introduction of the biological sample into the assay device results in a biological process by which fibrin and platelets may accumulate at a reaction zone of the assay device (e.g., both fibrin and platelets are capable of accumulating at the reaction zone). The assay device is capable of receiving one or more chemical reagents compatible with the biological sample and usable for detecting the accumulation of the fibrin and platelets within the reaction zone. The assay device is capable of receiving one or more drug reagents compatible with the biological sample and usable for modifying the accumulation of the fibrin and platelets within the reaction zone. The fibrin and platelets, and their associated signals, accumulated at the reaction zone of the assay device are usable to determine at least one of a drug presence, a drug class, a drug level in relation to a threshold, or a drug concentration, within the biological sample.

The system can include a fluorescent assembly capable of detection of the biological process by way of fluorescent labeling and detection of a resulting accumulating fluorescent signal. The system can include a processing device configured to receive as input a measurement of the fibrin and platelets to determine at least one of the drug presence, the drug class, the drug level in relation to a threshold, or the drug concentration, within the biological sample, and the processing device can be configured to correlate a measured fluorescent intensity with the accumulation of the fibrin and platelets in microfluidic flow paths of the assay device.

The one or more chemical reagents or the one or more drug reagents can be a fluorescent reagent capable of labeling the fibrin and platelets from the biological sample that results in a fluorescent assembly that reports the accumulation of the fibrin and platelets. The system can include a light source for monitoring clot development in microfluidic flow paths of the assay device and for detecting a fluorescent reaction of the one or more chemical reagents or the one or more drug reagents.

The assay device can include a first inlet port, a second inlet port, a third inlet port, and a fourth inlet port, each configured to receive the biological sample. The biological sample can be an unmodified sample including a platelet specific label and a fibrin specific label. The assay device can include an outlet port, and microfluidic flow paths fluidically connecting each of the first, second, third and fourth inlet ports with the outlet port. The introduction of the biological sample into the respective first, second, third and fourth inlet ports generates a fibrin signal and a platelet signal. At least one of the first, second, third and fourth inlet ports can be configured to receive an unmodified biological sample. At least one of the first, second, third and fourth inlet ports can be configured to receive a molar excess of drug to provide a fully attenuated fibrin or platelet signal.

At least one of the first, second, third and fourth inlet ports can be configured to receive a first reversal drug to identify a first class of drug present in the biological sample. At least one of the first, second, third and fourth inlet ports can be configured to receive a second reversal drug to identify a second class of drug present in the biological sample. The first reversal drug or the second reversal drug reverses effects of the drug or chemical that attenuates the fibrin or platelet signals in the biological sample to produce a fully recovered fibrin or platelet signal. The drug can be a direct-acting oral anticoagulant (DOAC), and the first and/or second reversal drug inhibits, antagonizes or attenuates the activity of a Xai or DTi class DOAC. The drug can be an anti-platelet medication, and the first and/or second reversal drug inhibits, antagonizes or attenuates an activity of the anti-platelet medication.

The system can include a processing device configured to manipulate the biological sample and monitor a direct response of the biological sample to the first and/or second reversal drug to identify the drug class present in the biological sample. The system can include a processing device configured to manipulate the biological sample and monitor a direct response of the biological sample to a molar excess of the fibrin or platelet attenuating drug to identify a drug or chemical level or concentration present in the biological sample.

The biological sample can include a raw blood sample, a processed blood sample, a blood sample treated with an anticoagulant to prevent intrinsic pathway coagulation activation, a citrated blood sample that is recalcified, a heparinized blood sample that is treated with protamine, or a blood sample treated with an antiplatelet drug. The system can include a processing device configured to compare the fibrin or platelet signal to the fully recovered fibrin or platelet signal, compare the fibrin or platelet signal to the fully attenuated fibrin or platelet signal, and compare all other coincident signals for all reactions to determine the drug presence, the drug class, the drug level in relation to the threshold, or the drug concentration, in the biological sample.

In some embodiments, the reaction zone can include a single flow path with separate clot sites in a serial configuration having different tissue factor (TF) concentrations. In some embodiments, the reaction zone can include two flow paths in parallel alone the same plane, each of the flow paths having different tissue factor (TF) concentrations. In some embodiments, the reaction zone can include two flow paths on separate planes of the assay device, the two flow paths each having a clot site in a non-overlapping configuration relative to each other and having different tissue factor (TF) concentrations. Increasing a reaction temperature at the reaction zone decreases initiation times, increases reaction rates, and provides a higher signal of the fibrin and platelet accumulation within the reaction zone.

In accordance with embodiments of the present disclosure, an exemplary method for drug or chemical detection and quantification is provided. The method includes adding a biological sample to an assay device, and adding one or more chemical reagents to the assay device to generate a biological process by which fibrin and platelets may accumulate at a reaction zone of the assay device. The method includes detecting the biological process with a fluorescent assembly by way of fluorescent labeling and detection of a resulting accumulating fluorescent signal, and using the fibrin and platelets accumulated at the reaction zone of the assay device, and the accumulating fluorescent signal, to determine at least one of a drug presence, a drug class, a drug level in relation to a threshold, or a drug concentration.

The method can include adding the biological sample to a first inlet port, a second inlet port, a third inlet port, and a fourth inlet port of the assay device. The assay device includes an outlet port and microfluidic flow paths fluidly connecting each of the first, second, third and fourth inlet ports with the outlet port. The biological sample can be an unmodified sample including a platelet specific label and a fibrin specific label. The method can include generating a fibrin signal and a platelet signal from the biological sample for each of the first, second, third and fourth inlet ports, and determining the drug presence, the drug class, the drug level in relation to a threshold, or the drug concentration from the fibrin signal and the platelet signal.

The biological sample can include a raw blood sample, a processed blood sample, a blood sample treated with an anticoagulant to prevent intrinsic pathway coagulation activation, a citrated blood sample that is recalcified, a heparinized blood sample treated with protamine sulfate, or a blood sample treated with an antiplatelet drug. In some embodiments, an unmodified whole blood sample may be preferred, although other blood samples can be equally valuable as well. In deference to common materials used for blood collection, citrated vacutainers, and lithium or sodium heparin vacutainers can all provide useful samples, provided the existing vacutainer anticoagulation is reversed prior to use in the assay. For example, citrate anticoagulated blood can be reversed to near normal coagulation levels with the addition of calcium. Heparin anticoagulated blood can be reversed to near normal coagulation levels through the addition of protamine sulfate. (See, e.g., FIGS. 48A-48B). Most reversibly anticoagulated samples can be utilized so long as the anticoagulation can be reversed quickly to a state that is similar to that of whole blood. By using the reversibly anticoagulated samples, useful results are still possible for modified blood samples. This helps resolve the issue of inadvertent collection of the blood sample into the “incorrect” blood collection device, and allows for additional time to test the sample since clotting is halted in anticoagulant treated samples, increasing working time.

The method can include identifying an overall state of coagulation from the unmodified sample by evaluating the fibrin signal and a fully recovered fibrin signal obtained using a reversal agent. The method can include identifying an overall state of platelet function from the unmodified sample by evaluating the platelet signal and a fully recovered platelet signal obtained using a reversal agent.

The method can include using a Xai reversal agent in the second inlet port and a DTi reversal agent in the third inlet port. The method can include receiving a concentration of a fibrin attenuating drug to a point which produces no further attenuation of the fibrin signal to obtain a fully attenuated fibrin signal. The method can include receiving a concentration of a platelet attenuating drug to a point which produces no further attenuation of the platelet signal to obtain a fully attenuated platelet signal. The method can include generating a platelet signal coincident with the fibrin signal for each of the first, second, third and fourth inlet ports. An unmodified sample fibrin signal and an unmodified sample platelet signal can be generated from a microfluidic flow path associated with the first inlet port, a fully reversed fibrin signal and a coincident platelet signal can be generated from either a microfluidic flow path associated with the second or third inlet port by way of interaction with a Xai or DTi reversal agent, and a fully attenuated fibrin signal and a coincident platelet signal can be generated from a microfluidic flow path associated with the fourth inlet port.

The method can include imparting a light source onto the microfluidic flow paths to monitor clot development in the microfluidic flow paths based on a fluorescent reaction of the one or more reagents, receiving as input at a processing device a measured fluorescent intensity of the monitored clot development, and correlating with the processing device the measured fluorescent intensity with platelet and fibrin accumulation in the microfluidic flow paths. The method can include comparing with a processing device the fibrin signal to a fully reversed fibrin signal, and a fully inhibited fibrin signal along with coincident platelet signals, to determine the drug class or drug concentration in the biological sample.

Any combination and/or permutation of embodiments is envisioned. Other objects and features will become apparent from the following detailed description considered in conjunction with the accompanying drawings. It is to be understood, however, that the drawings are designed as an illustration only and not as a definition of the limits of the present disclosure.

### BRIEF DESCRIPTION OF THE DRAWINGS

To assist those of skill in the art in making and using the system for DOAC-related data collection and analysis, reference is made to the accompanying figures, wherein:

FIG. 1A is a diagrammatic illustration of vasoconstriction with damage exposing collagen and tissue factor;

FIG. 1B is a diagrammatic illustration of a platelet plug formation with platelet aggregation;

FIG. 1C is a diagrammatic illustration of a clot formation in the form of coagulation;

FIG. 2 is a perspective view of a microfluidic device for monitoring blood biology under flow in an exemplary system for DOAC-related data collection and analysis;

FIG. 3 is a diagrammatic view of a microfluidic device for monitoring blood biology under flow in an exemplary system for DOAC-related data collection and analysis;

FIG. 4 is a flowchart illustrating a process of implementation of an exemplary system for DOAC-related data collection and analysis;

FIGS. 5A and 5B are diagrammatic views of a graphical user interface of an

exemplary system for DOAC-related data collection and analysis including results of the system;

FIG. 6 is a table of a sample data set relative to clinical decision points of interest;

FIG. 7 is a table of sample detection for a 30 ng/mL decision point;

FIG. 8 is a table of sample detection for a 50 ng/mL decision point;

FIG. 9 is a table of performance characteristics for Binary Logistic Regression and CART Classification models;

FIG. 10 is a graph of Binary Logistic Regression for sample detection of greater than or equal to 30 ng/ml;

FIG. 11 is a graph of CART Classification for sample detection of greater than or equal to 50 ng/ml;

FIG. 12 is a table of DOAC concentration prediction based only on a fibrin signal;

FIG. 13 is a table of DOAC concentration predictions based on both fibrin and platelet signals;

FIG. 14 is a graph of DOAC concentration predictions based on both fibrin and platelet signals using a CART Regression model verses LC-MS/MS measured data, specifically a CART regression model prediction using fibrin, platelet, fully reversed fibrin and fully reversed platelet as compared to an LC-MS/MS measured concentration of apixaban or rivaroxaban;

FIG. 15 is a graph of DOAC concentration predictions based on both fibrin and platelet signals using a Pearson correlation model verses LC-MS/MS measured data;

FIG. 16 is a table of patient data for apixaban and rivaroxaban;

FIG. 17 is a table of calibrators and controls for an LC-MS/MS assay for apixaban and rivaroxaban;

FIG. 18 is a graph of a fitted line plot for a fibrin signal with a time of maximum difference between the patient sample condition and the fully reversed patient sample condition;

FIG. 19 is a graph of a fitted line plot for a platelet signal with a time of maximum difference between the patient sample condition and the fully reversed patient sample;

FIG. 20 is a graph of a receiver operating characteristic (ROC) curve for Binary Logistic Regression for DOAC≥30 ng/mL including a fibrin signal;

FIG. 21 is a graph of a receiver operating characteristic (ROC) curve for Binary Logistic Regression for DOAC≥30 ng/mL including a fibrin signal, a platelet signal, a fully reversed fibrin signal, and a fully reversed platelet signal;

FIG. 22 is a graph of a receiver operating characteristic (ROC) curve for CART Regression for DOAC≥30 ng/mL including a fibrin signal;

FIG. 23 is a table of a confusion matrix for only a fibrin signal;

FIG. 24 is a graph of a receiver operating characteristic (ROC) curve for only a platelet signal for DOAC≥30 ng/mL;

FIG. 25 is a table of a confusion matrix for only a platelet signal;

FIG. 26 is a graph of a receiver operating characteristic (ROC) curve for only a fibrin signal for DOAC≥50 ng/ml;

FIG. 27 is a table of a confusion matrix for only a fibrin signal;

FIG. 28 is a graph of a receiver operating characteristic (ROC) curve for only a platelet signal for DOAC≥50 ng/mL;

FIG. 29 is a table of a confusion matrix for only a platelet signal;

FIG. 30 is a graph of a receiver operating characteristic (ROC) curve for both a fibrin and platelet signal for DOAC≥50 ng/ml;

FIG. 31 is a table of a confusion matrix for both a fibrin and platelet signal;

FIG. 32 is a graph of a receiver operating characteristic (ROC) curve for a fibrin and platelet signal, and for a fully reversed fibrin and platelet signal, for DOAC≥50 ng/ml;

FIG. 33 is a table of a confusion matrix for a fibrin and platelet signal, and for a fully reversed fibrin and platelet signal;

FIG. 34 is a graph of a matrix plot of LC-MS/MS and fibrin predicted concentrations;

FIG. 35 is a graph of a regression model prediction using fibrin, platelet, fully reversed fibrin and fully reversed platelet as compared to an LC-MS/MS measured apixaban or rivaroxaban concentration;

FIG. 36 is a graph of a matrix plot for an LC-MS/MS prediction based on a regression model;

FIG. 37 is a graph of a regression model prediction using fibrin, platelet, fully reversed fibrin and fully reversed platelet as compared to an LC-MS/MS measured apixaban or rivaroxaban using log10;

FIG. 38 is a table of coefficients for a constant and an LC-MS/MS;

FIG. 39 is a table of bins and LC-MS/MS measured apixaban or rivaroxaban concentration from the distribution of the LC-MS/MS results;

FIG. 40 is a histogram of LC-MS/MS measured apixaban or rivaroxaban concentration from the distribution of the LC-MS/MS results;

FIG. 41 is an IC50 curve for determination of drug concentration;

FIG. 42 is a diagrammatic view of microfluidic device flow path configurations for determining a presence of a single drug using a reversal agent in accordance with embodiments of the present disclosure;

FIG. 43 is a graph of “normal” platelet accumulation over time;

FIG. 44 is a graph of “normal” fibrin accumulation over time;

FIG. 45 are graphs of a subject result and a population dose response (IC50 curve);

FIG. 46 is a diagrammatic view of a coagulation pathway relating to the use of term DTi and IIai herein;

FIG. 47 is a diagrammatic view of a simplified version of the coagulation pathway of FIG. 46;

FIG. 48A is a chart of platelet signal change over time and FIG. 48B is a chart of fibrin signal change over time for protamine reversal of heparinized blood, with reversal of heparinized blood with protamine demonstrating recovery of fibrin and platelet signal to near normal levels with 0.1875 mg/mL of protamine;

FIGS. 49A-49D are charts demonstrating change in fibrin and platelet signal development in the presence of DOAC at two temperatures (FIG. 49A showing platelet signal at 37° C., FIG. 49B showing platelet signal at 21° C., FIG. 49C showing fibrin signal at 37° C., and FIG. 49D showing fibrin signal at 21° C.);

FIGS. 50A-50C are regression model charts, with FIG. 50A showing individual regression models for known drugs Apixaban (A), Dabigitran (D), and Rivaroxaban (R), FIG. 50B showing regression models with an unknown drug but a known drug class as the categorical input, and FIG. 50C showing CART regression models with unknown drug but a known drug class;

FIG. 51 is a diagrammatic view of microfluidic devices for determining a presence of a drug using a reversal agent in accordance with embodiments of the present disclosure;

FIG. 52 is a diagrammatic view of a microfluidic device pathway including a multi-layer structure with two unique reaction zones; and

FIG. 53 is a diagrammatic view of microfluidic device pathway including a single-layer structure with two unique reaction zones.

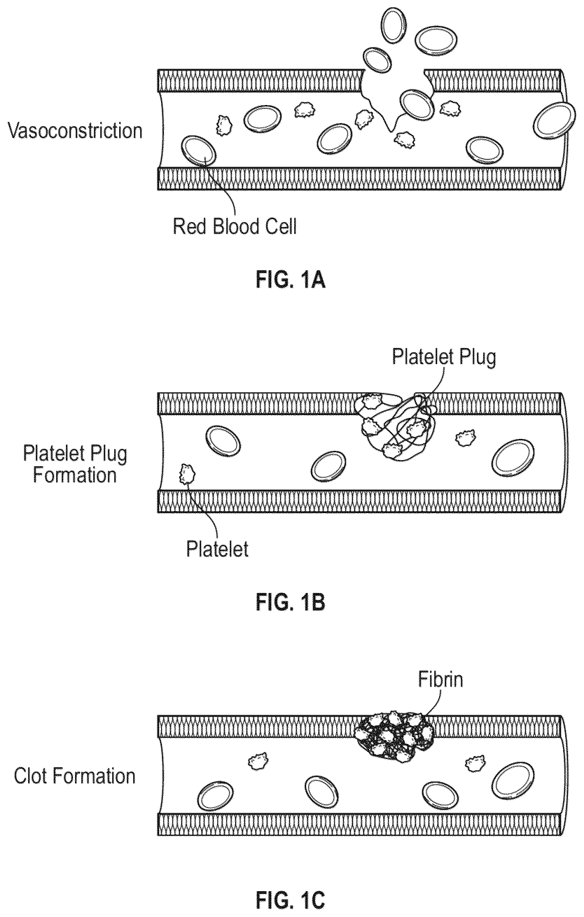

### DETAILED DESCRIPTION

The system and method for DOAC-related data collection and analysis provides a rapid and precise assessment of whether a patient is taking DOACs in a testing environment that is analogous to the blood clotting physiology found in the human body. The system performs a hemostasis assay that can be read from a portable tabletop instrument (analyzer) with an integrated computer, preformatted disposable cartridge and associated software. The system can be used to determine carly risk stratification, targeted treatment, and patient monitoring. The assay can mimic the microenvironment of blood clot formation, and incorporates hemodynamic flow and discrete clot activation. The system can provide real-time (or substantially real-time) characterization of blood clotting physiology under flow. The system can provide dynamic, multiplexed signaling of platelets and fibrin in a single test. The system can provide functional response and (estimated) drug levels. The system can provide rapid results (e.g., over the course of 15 minutes or less) in real-time or substantially real-time, and provides a comprehensive determination of hemostasis function and anticoagulation levels in a single test, within 30 minutes. Such determination can be essential in emergency critical are settings, as well as other patient treatment settings, and assists with reversal and monitoring strategies. In addition, the system could provide additional information about clotting behavior, if analyzed over longer periods of time (e.g., fibrinolysis and platelet inhibition, or the like).

The system simulates in-vivo conditions and therefore is sensitive to the clotting behavior of the patient. The information/results provided by the system can include independent functional fibrin and platelet response, drug classes, levels and/or concentrations, and clotting behavior over time. These results can distinguish platelet and coagulation dysfunction, as well as a drug level and/or concentration associated with the dysfunction. The system can facilitate understanding the effects of blood product delivery, e.g., blood transfusion, fresh frozen plasma (FFP), cryoprecipitate, platelets, or the like, as well as real-time monitoring of drug action. The system can specifically fill the DOAC information gap by detecting and classifying the DOAC, predicting DOAC levels and/or concentrations, and whether a need for reversal exists, supporting the evaluation of targeted reversal strategies, and blood product stewardship.

The system can use the same drug(s) that the patient is prescribed or treated with to determine drug presence and class using multiple reversal agents. With respect to the drug and its reversal agent, the system can simultaneously use the patient's own blood as an internal control to determine DOAC drug activity, avoiding calibration requirements of some traditional assays (e.g., the patient's own blood is used to determine the response to the reversal agents, as well as its response to full inhibition, to avoid the need for a calibration curve for every device run). Specific targeting clements at the reaction zone facilitate clotting that involves both platelet accumulation and fibrin formation, coincidently, regardless of the detection reagent. The combination of the fibrin signal and platelet signal has been shown to both be meaningful to calculating drug presence and concentration. Additional targeting elements can be added to the reaction zone to target additional clotting elements, hemostatic elements, and/or other blood born factors. In some embodiments, clotting can occur in the presence of whole blood that contains all of the necessary components for hemostasis that is a direct reflection of the individual patient's in-vivo processes.

In some embodiments, the reaction zone can be “tuned” towards different levels of sensitivity for platelets and fibrin accumulation by varying the specific targeting elements (e.g., tissue factor) and concentrations, modifying the shear rate, modifying the reaction temperature, or modifying the fluidic dimensions, thereby allowing for specific levels of sensitivity to drug levels. The reaction zone contains immobilized reagents which bind and activate the platelets and activate coagulation. In some embodiments, more than one reaction zone with more than one level of sensitivity could be placed on the same assay device, thereby broadening the ability of the device to measure a wider range of drugs. Blood can be analyzed under physiological flow, coincidently, thereby demonstrating the actual likely behavior of the patient's blood makeup (platelets and fibrin) in vivo. In some embodiments, blow food can be driven through the device by an external source (e.g., a pump) to achieve a specific range of physiological shear conditions. For example, the biological sample flow can be driven through the assay device from the inlet across the reaction zone and to the outlet via application of a pressure gradient using the external source.

Temperature Control and Effects on Assay Results: For a kinetic/time based assay, temperature can play a large role in the behavior of individual blood components (such as the serine protease thrombin). Reactions run at room temperature, some 17 degrees Celsius below body temperature, can have substantially reduced activity (reaction kinetics), as well as increased blood viscosity. A consistent reaction temperature is critical in terms of comparing results across time, location, and sample, to avoid variance caused by differences in the system's environment. While it is possible to analyze blood at lower than body temperatures, the behavior of blood becomes less active the lower the analysis temperature. This can be advantageous to the design of a system that utilizes this effect, as temperature can be used to control or modulate both platelet and coagulation behavior (e.g., reducing or increasing overall signal or rate of signal development, reducing or lengthening the overall assay time and/or modifying assay sensitivity).

The addition of temperature control to the assay can be done most simply through the controlled heating of the system's imaging location. For example, heating elements can be applied to the floor of the imaging area allowing (indirect) convection heat transfer to control the temperature of the space where the fluidic device is being imaged. Enclosing and insulating the imaging/reaction space can provide for reduction in temperature fluctuations. Alternatively, heating elements within the imaging/reaction space can be brought into contact with the bottom of the fluidic device when inserted into the system, to directly heat the device. In some embodiments, a combination of direct and indirect heat transfer can be used. A thermocouple or similar type temperature sensor can be used as a feedback mechanism to control the temperature to a specific setpoint, at, above or below body temperature. In some embodiments, one or more heating elements can be directly applied to, or within, the fluidic device, whereby once placed into the instrument, a connection to the instruments electrical system can supply power to the heating element(s) within the fluidic cartridge. Multiple ways of delivering and applying heat (e.g., direct contact, air, liquid, combinations thereof, or the like) and its control (PID, hysteresis, or the like) to the fluidic device within the instrument are contemplated. In some embodiments, cooling of the imaging/reaction chamber can be applied by means of, e.g., a Peltier device, or the like, to reduce reaction temperatures (and therefore assay activity) if needed.

Applying a consistent temperature to the assay can result in either increased reactivity/signal generally with increasing temperature, and decreased reactivity/signal generally with decreasing temperature. This is advantageous in that sample reactivity, and potentially even sample sensitivity, can be modified simply by way of reaction temperature changes. For example, increasing reaction temperatures can decrease assay time, thereby shortening the length of time to reach a clinical result, which is critical in emergency critical care decision making. Increasing the reaction temperature can increase reaction kinetics for samples that are less reactive. For example, for patients on very high doses of a DOAC drug, the determination of drug concentration can be more easily discerned if the reaction kinetics are increased by increasing temperature. This can result in improving the assay's sensitivity to higher drug levels; or conversely, making the assay more sensitive to lower drug levels, using lower temperatures. In addition, reducing the temperature can help in evaluating hyper-reactive samples (e.g., hyper coagulation, excessive platelet activity, or the like). Hyper-reactive samples (either coagulation or platelet function) can cause excessive clotting within the device which can lead to fluidic blockage and spurious (or no) assay results. For example, seriously ill (e.g., COVID-19, sepsis, cancer, or the like) patients can present with hyper-coagulation issues. Reducing the reaction temperature of the patient's sample can reduce reaction kinetics sufficiently to avoid early device blockage and allow for useable clinical results.

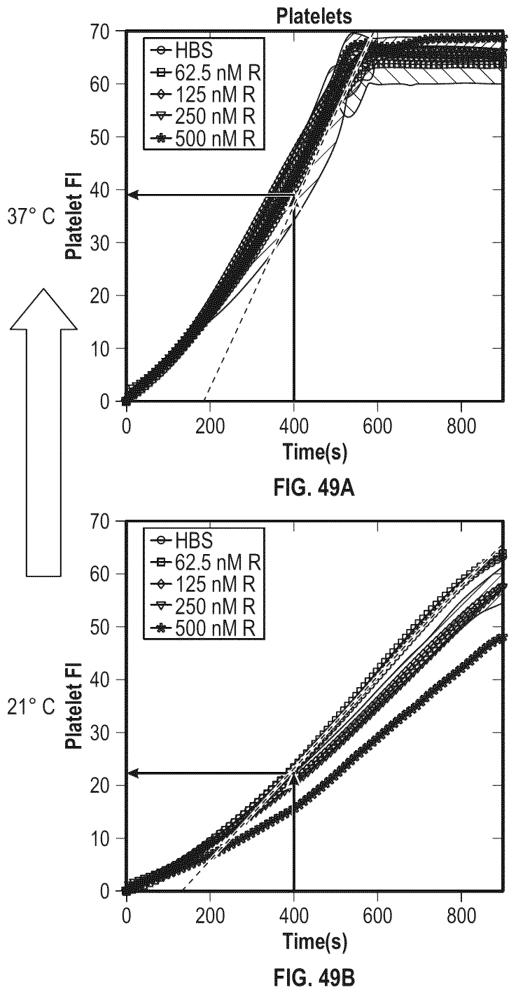

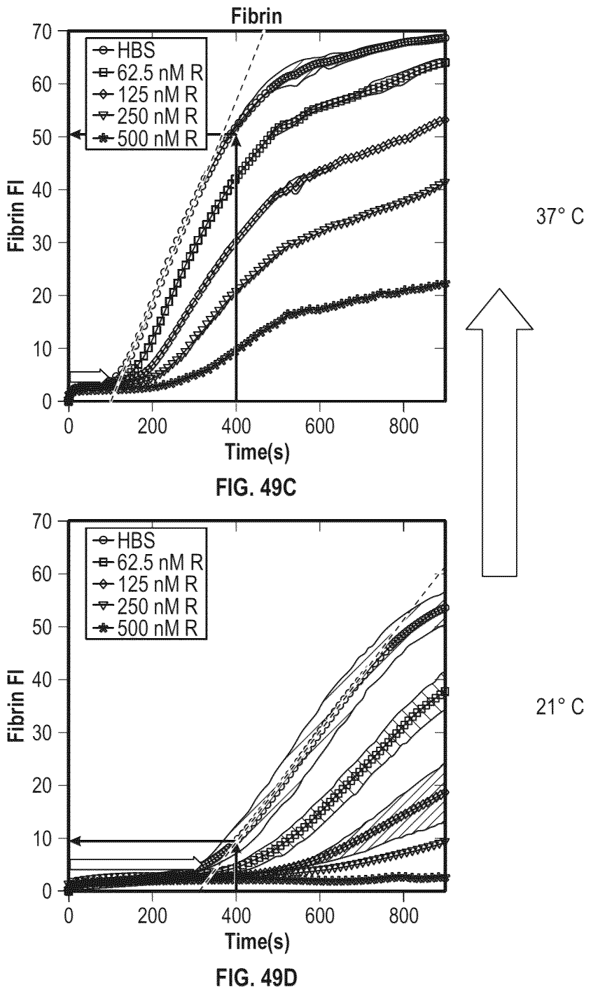

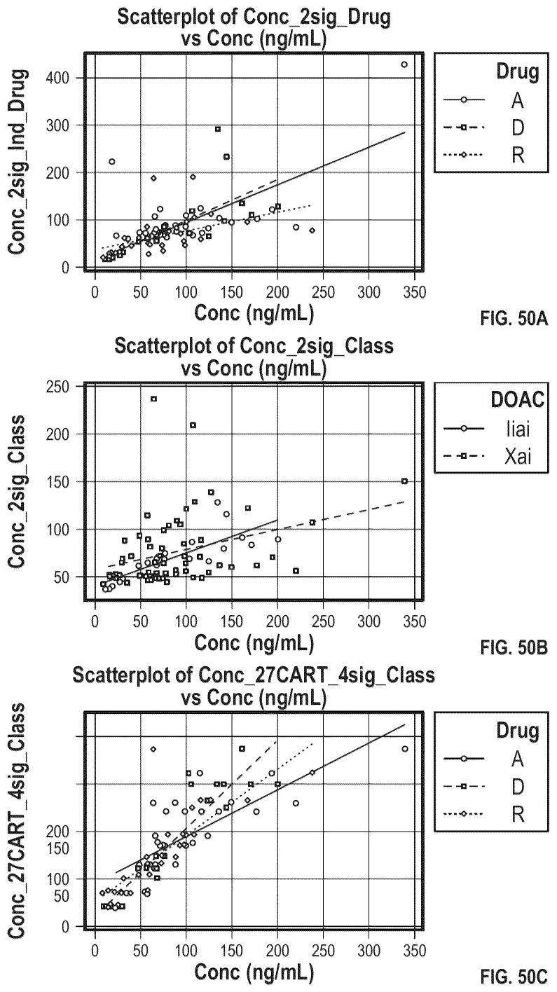

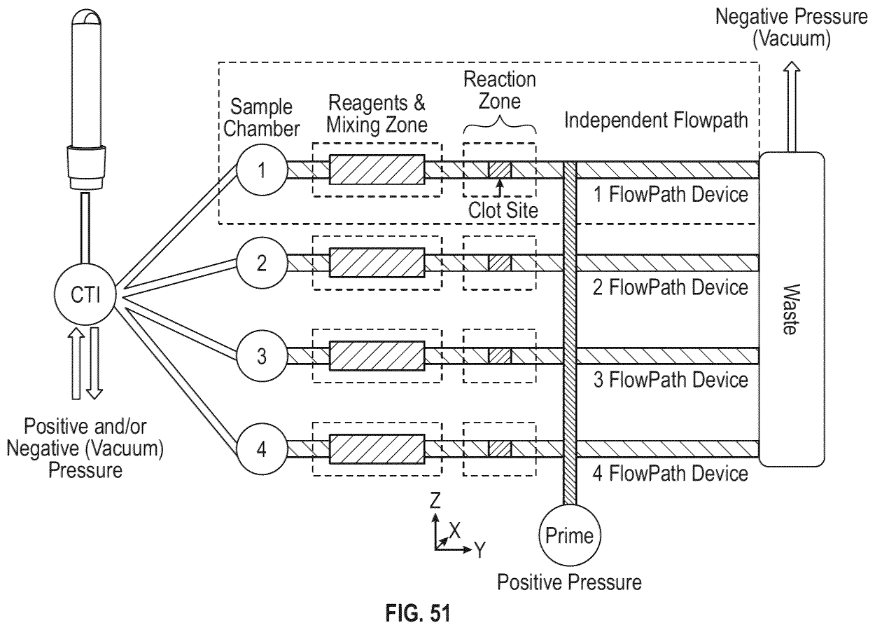

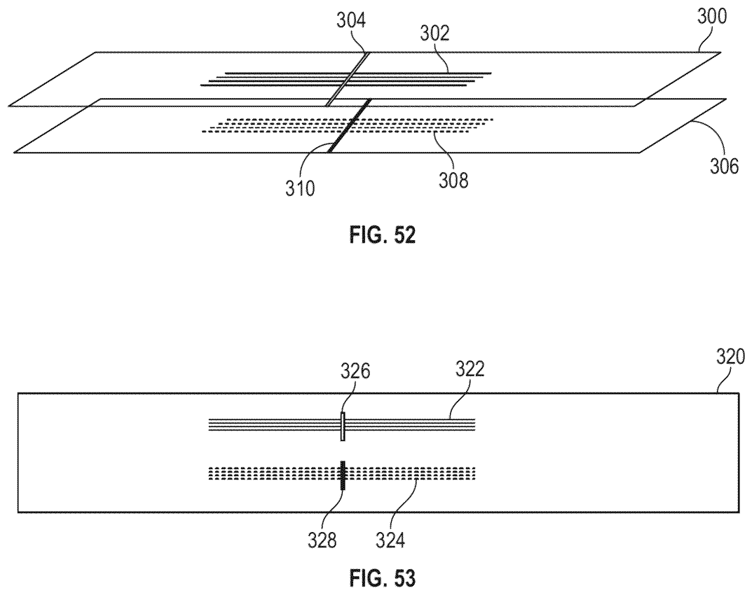

FIGS. 49A-49D demonstrate that in a blood sample treated with different doses of Rivaroxaban, the same overall dose specific fibrin behavior is seen, but with decreased initiation times, increased reaction rates, and higher signal as reaction temperature is increased, relative to body temperature. In particular, FIGS. 49A-49D demonstrate changes in fibrin signal development in the presence of DOAC, at two temperatures. Fibrin signal decreases as Rivaroxaban (R) dose is increased, in relation to the HBS control, as expected. As temperature increases, fibrin reactions initiate carlier, fibrin and platelet maximum rate of change increases (steeper slope), and fibrin and platelet signals are increased overall at the same point in time. The DOAC drug dose effect seen at higher temperatures follows similar dose behavior seen at lower temperatures. As illustrated in FIGS. 49A-49B, about a two-fold platelet signal increase at 400 s is seen, a substantial increase in maximum rate of change of fluorescence is also seen. As illustrated in FIGS. 49C-49D, about a five-fold fibrin signal increase at 400 s is seen, more than 3× faster initiation, and a substantial increase in rate of signal development is also seen.

The assay device can use flow channels (microfluidics) that mimic physiological structure, function in size, flow and distribution of cellular and subcellular components, and/or mimicking platelet margination effects under flow. The assay can measure the biological process of hemostasis in real-time, thereby allowing for assessment of the process at all time points during the assay (e.g., a kinetic assay). The system is rapid, providing a clear picture of hemostatic function within 15 minutes (in most cases). The system has a long term possibility of measuring clot lysis, clot strength, hematocrit, and other parameters of interest that affect hemostasis coincidently with platelet and fibrin function. The system can detect classes of drugs that affect both platelets and fibrin.

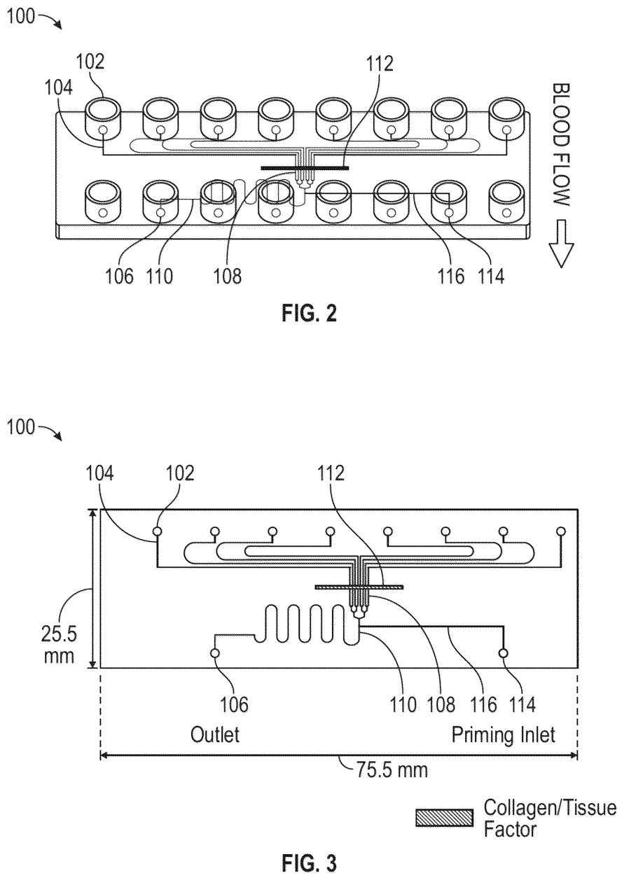

FIG. 2 is a perspective view of a microfluidic device 100 for monitoring blood biology under flow in the exemplary system, and FIG. 3 is a diagrammatic view of the microfluidic device 100. As such, the same reference numbers are used to represent the same structures. The device 100 generally includes microfluidic circuits including multiple inlet ports 102 (e.g., 8 inlet ports), a microfluidic flow path 104 in fluidic communication with each of the inlet ports 102 and leading to a single outlet port 106. In some embodiments, the device 100 can include a single dedicated inlet port for each of the respective inlet ports 102 In some embodiments, the device 100 can include dedicated outlet ports for each of the respective inlet ports 102. The flow paths 104 can all converge, creating an imaging zone 108, before a flow path 110 extends to the outlet 106. In some embodiments, the flow path 110 can be substantially linear. In some embodiments, the flow path 110 can define a serpentine or continuously curving configuration. Critically, all flow paths should be of the same length between inlet and imaging region (and imaging region to outlet), to ensure that resistance to flow is consistent between flow paths, and therefore, for a given applied pressure, the flow rates are identical as well. It should be noted that flows paths of different lengths could be utilized so long as the resistance to flow is balanced between the flow paths. This can be done by several means, including reducing the overall size of the flow paths in specific areas to increase resistance to flow, or by expanding the flow path size in specific areas to reduce resistance to flow.

The device 100 can include a collagen/tissue factor reaction zone 112 positioned at or near the imaging zone 108 of the device 100. Bringing the fluidic paths in proximity to one another simplifies imaging by allowing for imaging of all flow paths/reaction zones within the same field of view (although imaging using multiple fields of view using higher magnification would also be beneficial in some instances). Additional reactants specific to clot formation are also expected to be useful in the reaction zone (e.g. Factor XIa, Kaolin, collagen related peptides, or the like). The imaging zone can also include fiducial marks (both fluorescent and non-fluorescent) that allow for detection and alignment of the reaction zone automatically via the instrument's software.

A priming inlet 114 can be in fluidic communication with a microfluidic priming flow path 116 that connects in a fluidic manner with the outlet flow path 110 at or near the imaging zone 108. In operation, when a priming fluid is applied under pressure to the priming circuit, the priming fluid can flow through the microfluidic flow path 104 to the inlet ports 102 due to low resistance to flow in the microfluidic flow path 104 relative to the outlet 106. Other devices and methods of priming are envisioned, such as those described in International Patent Application No. PCT/US2019/022965, which is incorporated herein by reference in its entirety.

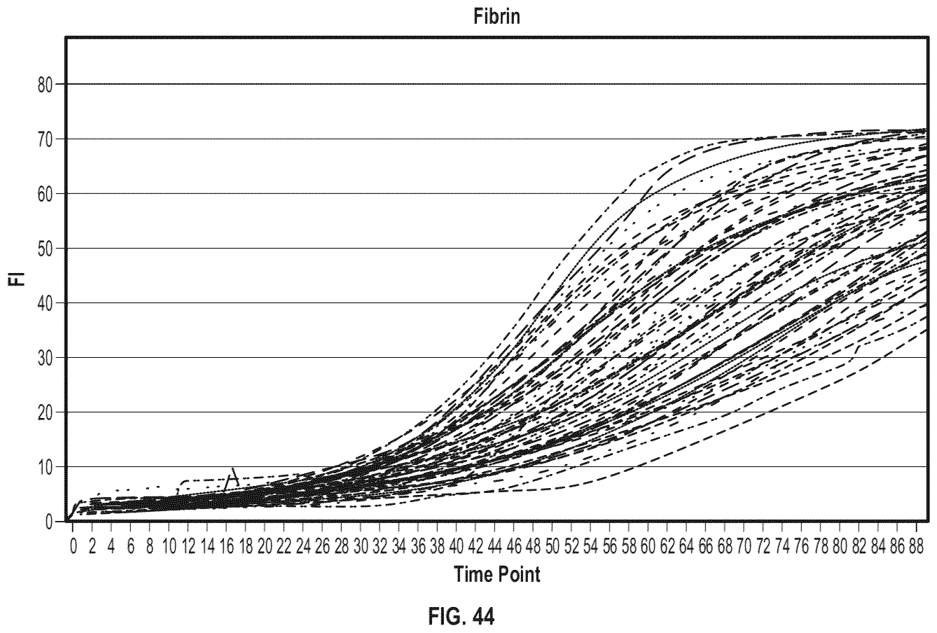

The device 100 can allow for up to eight unique conditions to be tested simultaneously. Flowing blood over a localized region of collagen and tissue factor 112 can induce clot formation (platelet and fibrin deposition) and allows for sensitivity to any disruption of the hemostasis/coagulation process. The assay test using the device 100 provides the ability to detect DOAC type by using the actual reversal drug. Estimated drug level can be determined through comparison to a fully attenuated sample and a fully reversed sample, in comparison to an IC₅₀ drug response curve, linear regression, CAR-T analysis or other statistical methodology. The DOAC test can use Factor Xa inhibitor at maximal inhibition, Factor Xai reversal agent (complete reversal), Direct Thrombin inhibitor (DTi) reversal agent (complete reversal), and unmodified patient blood. For example, two inlet ports can receive the maximum inhibition (high dose), two inlet ports can receive the Factor Xai reversal, two inlet ports can receive the Dti reversal, and two inlet ports can receive the unmodified patient blood. The utility of using multiple inlet ports with the same reactants is to allow for averaging of results, that can mute the effects of variability in device performance, and preparation. The reaction zone 112 can have one, two or multiple collagen/tissue factor regions for evaluating clotting function (sec, e.g., FIG. 44). These reactants can be the same in concentration and makeup (useful for statistical/averaging purposes) or could utilize varying concentration and makeup to evaluate different sensitivities of the sample to coagulation and platelet function. In some embodiments, one or more inlet ports could be used to determine other blood characteristics, such as hematocrit, oxy-hemoglobin, platelet count, or similar results at the reaction zone, or some other location on the device, within a flow path, using published means.

The system can rely on direct fluorescence signals for analysis of the results at the collagen/tissue factor 112. Fresh whole blood (used within minutes of venous draw), AF594 fibrinogen label (fibrinogen from human plasma, Alexa Fluor™ 594 conjugate) and platelet MCA2588A488 label (mouse anti-human CD61, Alexa Fluor™ 488 conjugate) can be added to the device 100 and the direct fluorescence signals can be analyzed. Chemicals that inhibit specific coagulation pathways, such as corn trypsin inhibitor or PPACK, can also be added to the patient blood (either before or along with the labeling chemicals) to prevent unwanted reactions, such as contact activation of the intrinsic coagulation pathway. The result is the production of direct fluorescent signals in green (platelets) and red (fibrin) where the measured fluorescent intensity (FI) directly correlates with the accumulation of platelets and fibrin. The fluorescent signal intensity can be directly measured at each clot site over time to produce clot response curves for both fibrin and platelets. Fluorescence signal (intensity) can be extracted for each clot and plotted for each assay condition.

The platelet and fibrin label can be added and mixed with the blood prior to addition to the inlet ports 102, or could be added to the inlet ports prior to adding and mixing the blood. It is contemplated that the reagents could also be stored wet or dried/lyophilized within the inlet ports 102 (or other locations within the device) before use. The addition of the blood and mixing of the reagents can be performed manually or automated (pipetting robot) off the device, or could be performed within the device using macro and microfluidic structures known to those in the art.

The determination of DOAC class can be performed by comparing the change in biological response between the unmodified sample, and a sample modified with reversal agent. DOACs are generally provided in two classes—Factor Xa inhibitor (Xai) and Factor IIa (thrombin) inhibitor (IIai or DTi), both of which affect fibrin formation. By using small amounts (micrograms) of the actual reversal agent used in-vivo to facilitate the reversal of Xai or IIai DOAC activity in the blood (the actual medically prescribed reversal agent is used with the assay device), the assay device directly identifies which DOAC class is present by the direct coagulation response of the patient's blood. For example, when a Factor Xai is present in the patient's blood, ANDEXXA® (Xai reversal agent) would improve the clotting behavior (increase fibrin signal) while PRAXBIND® would not. Similarly, when a Factor IIai is present in the patient's blood, PRAXBIND® (IIai reversal agent) would improve clotting behavior (increase fibrin signal) while ANDEXXA® would not. If neither DOAC class was present in the sample, neither reversal agent would have a significant effect on the fibrin signal (no specific increase nor decrease would be seen).

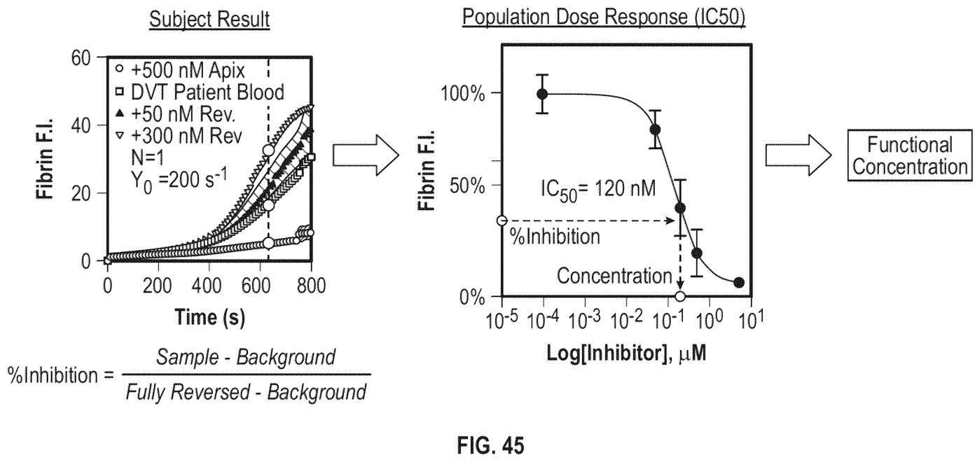

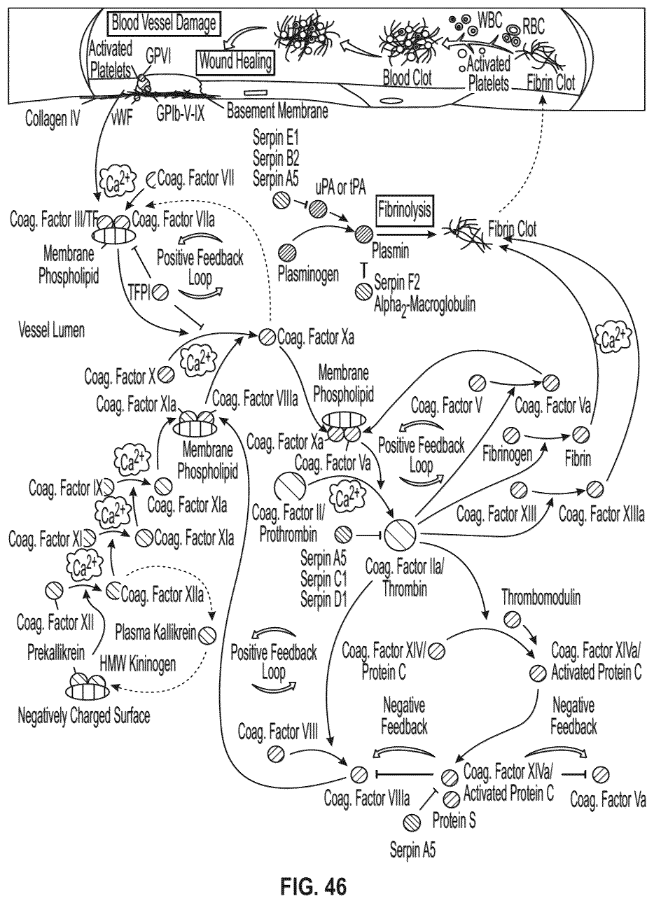

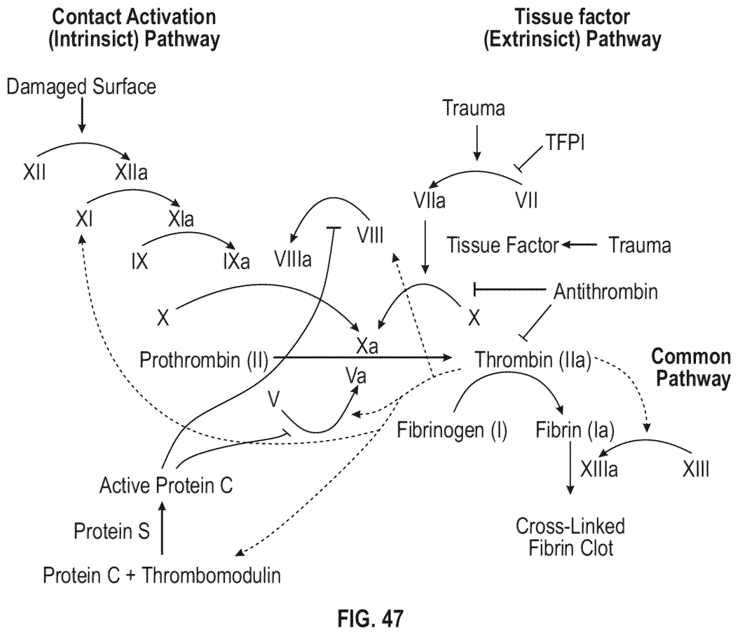

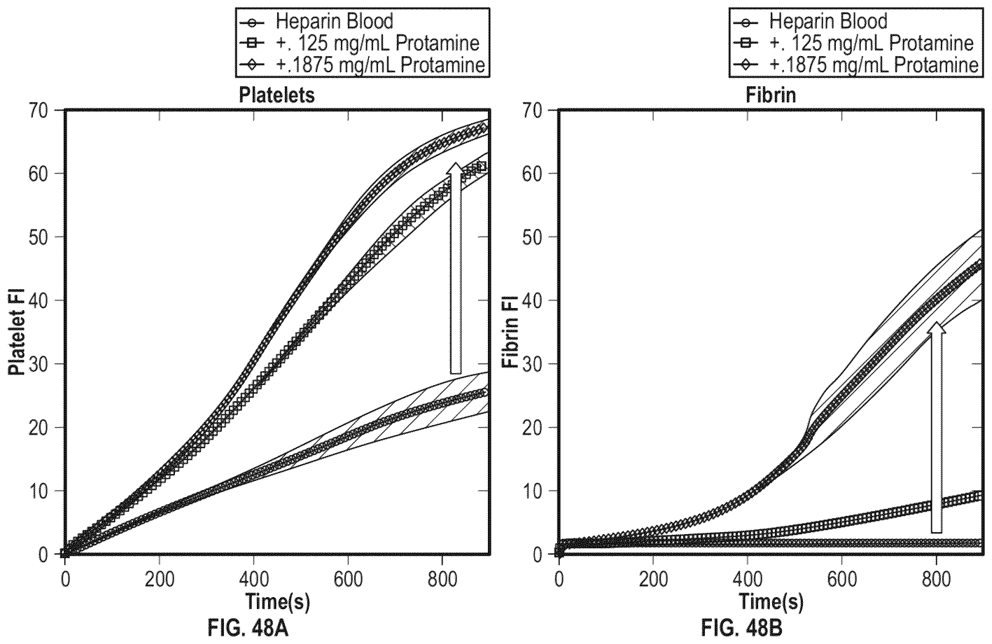

The determination of the DOAC concentration can be determined by comparison to a population averaged dose response that is specific to the drug being evaluated. Because DOACs specifically affect fibrin formation through their direct or indirect reduction in thrombin activity, the ratio of the patient's fibrin signal to the patient's fully attenuated fibrin signal, and fully reversed fibrin signal, can be used to determine the approximate drug dose from a known IC₅₀ drug response curve, developed using the device/system. Each drug (or class) in question would have its own IC50 curve and comparison of the patient data would be specific to the drug identified in the sample. Data can be normalized to the patient's own coagulation response (the unmodified sample) (see, e.g., FIG. 45 illustrating the subject result and the population dose response using the IC50 curve).

The exemplary system/device can also rely on a statistical method for deriving the drug concentration from a comparison to results from a quantitative analytical technique, such as LC/MS. This method does not necessarily require knowledge of the specific drug in question, as the derivation of the result is dependent upon the relationship between the output of the device and the independent quantitative measurement. In this case, only four signals (two for fibrin function and two for platelet function) are needed to derive a result. For example, the device can include a first inlet port for providing the unmodified sample fibrin and platelet signal, and a second and third inlet port to evaluate fibrin and platelet signal reversal in the presence of a DOAC. The fibrin and platelet signal in a fourth inlet port containing the fully DOAC inhibited sample may also provide additional analytical benefit, such as verifying that the patient's blood behaves as expected when a coagulation inhibitor is present. In some embodiments, the fourth inlet port data can be used to assess acceptable system performance. When a DOAC is detected, the unmodified and reversed platelet and fibrin signals can be compared to determine the class of DOAC present, and the concentration of DOAC (e.g., quantitation of DOAC). Rather than relying only on the fibrin signal, the system relies on both unmodified and fully reversed signals for fibrin and platelet function to provide a more precise output of classification and quantification. These four signals are generated by the device/system simultaneously (or substantially simultaneously). The class of DOAC present can be determined by the system by a simple comparison (ratio) between the unmodified sample and the fully reversed samples. Regression and/or classification and regression tree (CART) analysis can be performed by the system based on the four signals to determine the DOAC concentration. The level of the detected DOAC can be compared with industry thresholds to provide guidance to the medical professional regarding next steps of patient treatment, e.g., reversal.

The system can include associated software on a computing or processing device capable of controlling/monitoring use and analysis of the assay device and the data generated by the assay device. In some embodiments, the computer is separate from the imaging device, while in other embodiments, the computer is built within the device. The software can control instrument functions, such as camera gain, frame rate, light emitting diode (LED) intensity, pump action, and pressure control, as well as other electro-mechanical functions. Images of clot formation, as well as plotted data extracted from those images can be shown in real-time, or substantially real-time.

Blood can be drawn from the patient and into a vacutainer or other suitable holding device, such as a syringe. Blood could alternatively be drawn directly into the device through an appropriate connection (e.g. an IV line and Leur connection) The vacutainer can be plugged into the device, and the device can be loaded into an analyzer (e.g., an imaging instrument with computing and software for analyzing the device). Automated analysis can provide results to the medical professional in 15 minutes or less. Useful data from the process of analyzing a sample may be discernible at earlier time points, including as early as 30 seconds into the run. A readout/report can be generated from the data and provided to the clinician. A user interface on a computer or mobile device can be used to visualize the data in real-time, or a report with information relating to the assay device testing and analysis, e.g., fibrin, platelet, and DOAC information. Additional useful data from the process may be attainable 30 minutes or later after initiation of the analysis (e.g., clot lysis).

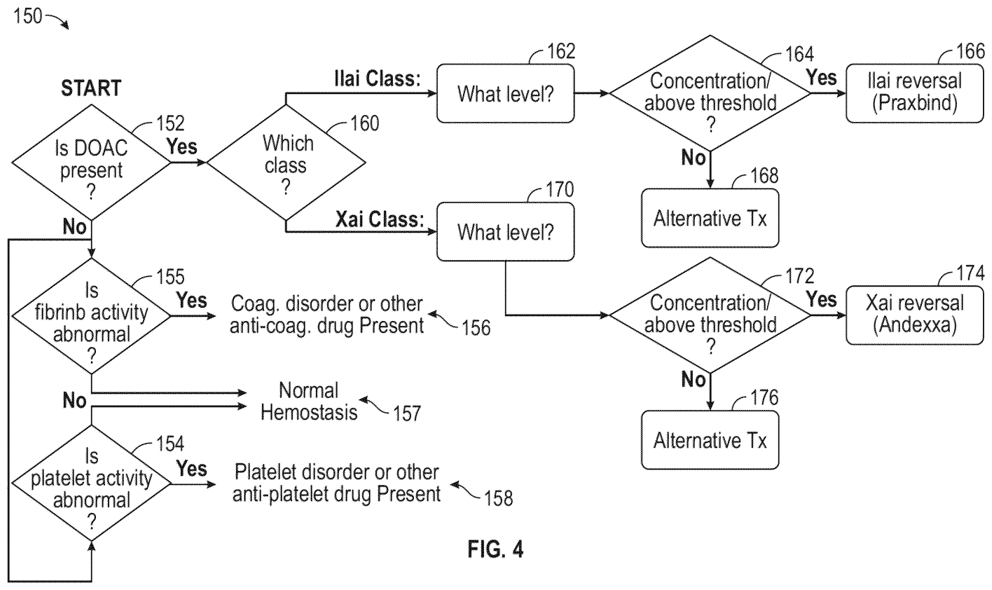

FIG. 4 is a flowchart illustrating a process 150 of implementation of the exemplary system discussed herein. At step 152, the system can be used to determine if DOAC is present in the patient's blood sample. If no DOAC is detected, at steps 154, 155, the system determines if the platelet and fibrin activity is abnormal. If the platelet and fibrin activity is not abnormal (i.e., normal), at step 157, normal hemostasis can be identified. If the fibrin activity is abnormal, the medical professional is notified to inquire about a coagulation disorder or other anti-coagulation drugs present (step 156). If the platelet activity is abnormal, the medical professional is notified to inquire about a platelet disorder or other anti-platelet drugs present (step 158). At step 160, if DOAC is detected, the system can be used to determine which class of DOAC is present.

At step 162, for the IIai class, the system can be used to determine what level of the DOAC is present. At step 164, the system can be used to determine whether the concentration level is above or below a threshold value. The system can direct the medical professional to initiate a IIai reversal if the DOAC level is above a threshold concentration value (step 166) or an alternative Tx procedure if the DOAC concentration level is below a threshold value (step 168). For the Xai class, at step 170, the system can be used to determine what level of the DOAC is present. At step 172, the system can be used to determine whether the concentration level is above or below a threshold value. The system can direct the medical professional to initiate an Xai reversal if the DOAC concentration level is above a threshold value (step 174) or an alternative Tx procedure if the DOAC concentration level is below a threshold value (step 176). Based on the output results of the DOAC class and level, the medical professional/clinician can make decisions regarding reversal therapy and next treatment plans. The assay device can verify if a DOAC is the cause of delayed or weak coagulation, or can indicate if dysfunction may come from another source. Results from the system can be coordinated with other blood tests (e.g., platelet count) to verify hemostasis and coagulation function of the patient.

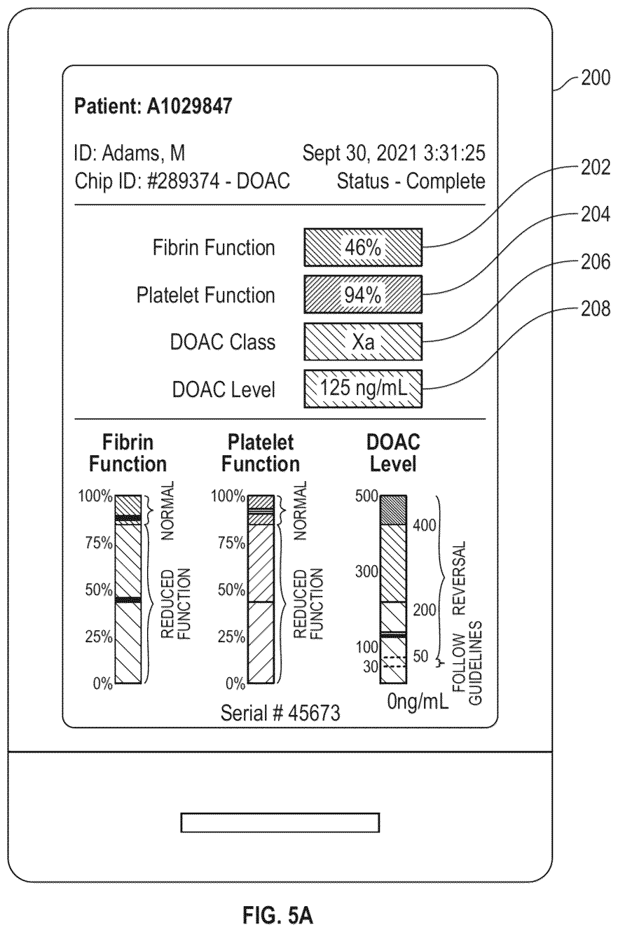

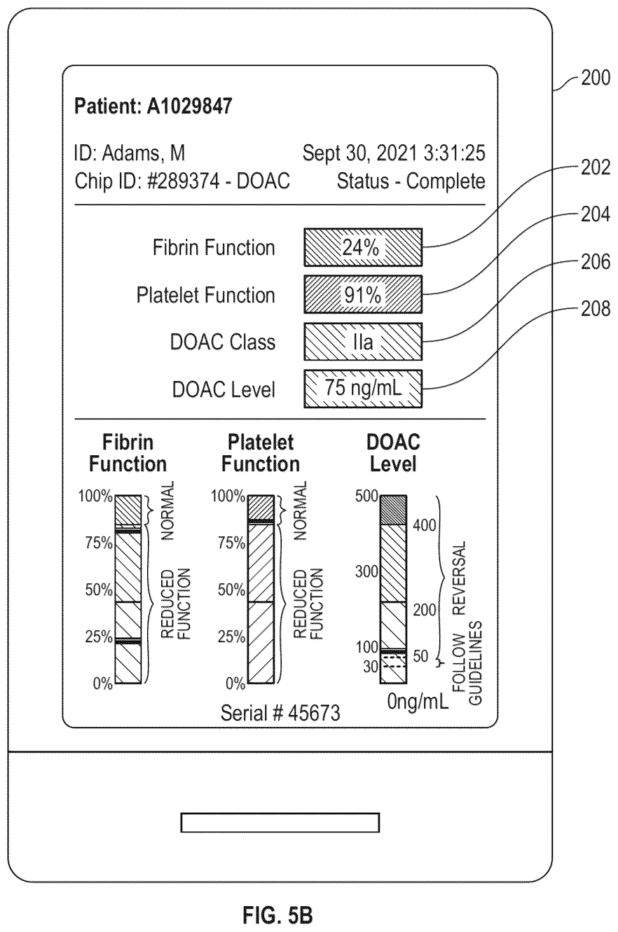

FIGS. 5A and 5B are diagrammatic views of a graphical user interface 200 of the exemplary system for displaying results to the clinician based on the automated analysis and determination of the assay device and associated software. The interface 200 can display the fibrin function in section 202, the platelet function in section 204, the DOAC class detected in section 206, and the DOAC level in section 208. The user interface 200 can thereby provide a simple readout of the DOAC class detected (or not), the estimated DOAC concentration in the patient's blood (if present), the patient's fibrin response to a DOAC drug and reversal agent (if DOAC was present), and the overall platelet function, in comparison to population norms.

Experimentation was performed with the exemplary assay device and system. In particular, testing was performed on and data was gathered from samples from patients taking DOACs using a microfluidic method with fresh whole blood and independently tested using plasma derived from those patient blood samples, by Pharmaron ((PH)—a commercial analytical services company), with LC-MS/MS. The testing included 13 patients taking apixaban and 7 patients taking rivaroxaban. The testing configuration used an eight channel microfluidic device (e.g., the exemplary assay device) with four different conditions, each in duplicate. The four different conditions included (i) an unmodified patient sample, (ii) a patient sample with DOAC 5000 nM clotting inhibition, (iii) a patient sample with DOAC partial reversal, and (iv) a patient sample with DOAC fully reversed. In some instances, the amount of reversal agent used may result in an incomplete reversal of fibrin signal. Intermediate concentrations of reversal agent can be used to distinguish intermediate concentrations of DOACs on board the patient by the incomplete reversal of fibrin signal.

Two signals were generated in the test as fluorescence intensity (FI) for fibrin and platelets accumulating in the region of interest on the device. The average of the duplicates in each of the four channel conditions for fibrin and platelet signals was taken, and there were a total of eight signals available for data analyses. The optimal time at which to select data for analyses was determined to be the time of maximum difference between the unmodified patient blood signal and the fully reversed signals using fibrin FI. This is referred to as the “time of max A” and ranges from 300 to 1520 seconds (5 to 25 minutes). In some embodiments, the time range can be between about, e.g., 1-25 minutes, inclusive, 1-5 minutes inclusive, 5-25 minutes inclusive, 1-60 minutes inclusive, 5-60 minutes inclusive, 25-60 minutes inclusive, or the like. Comparisons of the exemplary assay device testing results versus the LC-MS/MS results are provided on a ng/ml basis (consistent with clinical decision making for DOAC reversal). A variety of analyses were performed. The highest performance of the exemplary assay device testing versus LC-MS/MS is discussed herein.

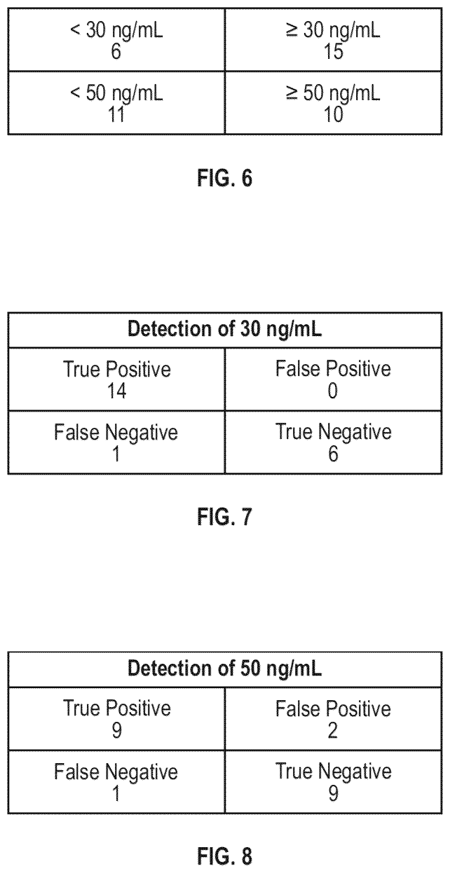

During experimentation, the two clinical decision points of interest were 30 ng/ml and 50 ng/mL. FIG. 6 is a table of a sample data set relative to clinical decision points of interest. In particular, in the 21 sample data set, the numbers of samples either less than or equal to or greater than these values are illustrated in FIG. 6. Two methods of classification provided the best performance using four of the eight possible signals available. The four signals that provided the best performance were (i) unmodified patient sample with DOAC, fibrin FI, (ii) unmodified patient sample with DOAC, platelet FI, (iii) patient sample with DOAC and, fibrin FI of fully reversed sample, and (iv) patient sample with DOAC and, platelet FI of fully reversed sample.

These four signals were used as inputs to different methods for categorical discrimination around the clinical decision points of 30 ng/mL and 50 ng/ml. One method was fitted to a Binary Logistical Regression model, and the other CART Classification. Binary Logistic Regression provided the best performance for the 30 ng/ml decision point, and CART Classification provided the best performance for the 50 ng/mL decision point.

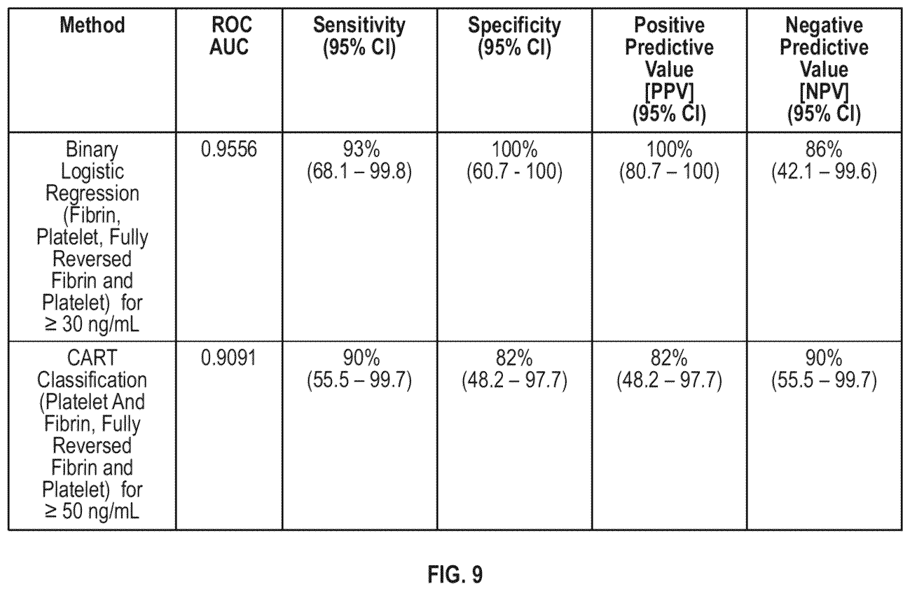

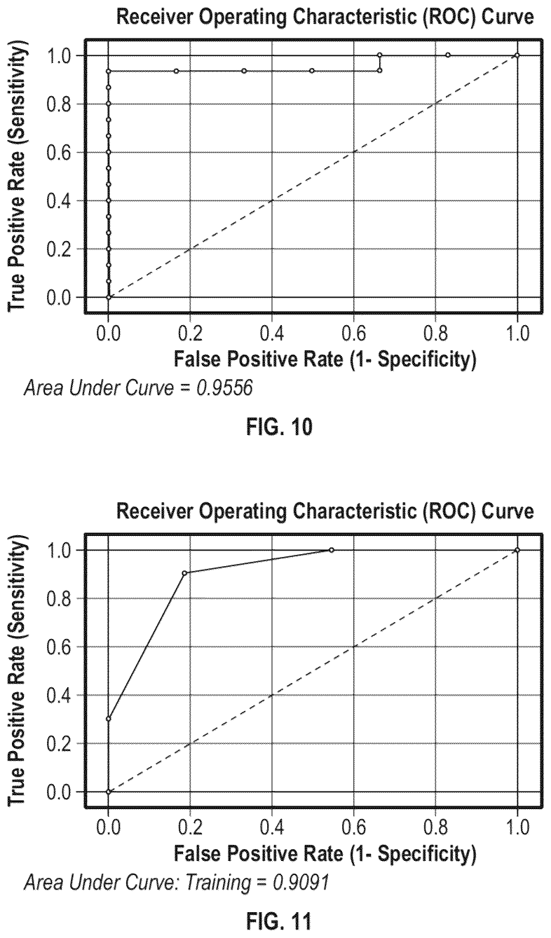

Test performance is provided in the tables of FIGS. 7-9 (e.g., “truth tables”), and the associated performance characteristics. In particular, FIG. 7 is a table of sample detection for a 30 ng/ml decision point, FIG. 8 is a table of sample detection for a 50 ng/mL decision point, and FIG. 9 is a table of performance characteristics for Binary Logistic Regression and CART Classification models. FIGS. 10 and 11 are graphs of the performance for discrimination. In particular, FIG. 10 is a graph of Binary Logistic Regression for sample detection of greater than or equal to 30 ng/ml, and FIG. 11 is a graph of CART Classification for sample detection of greater than or equal to 50 ng/mL.

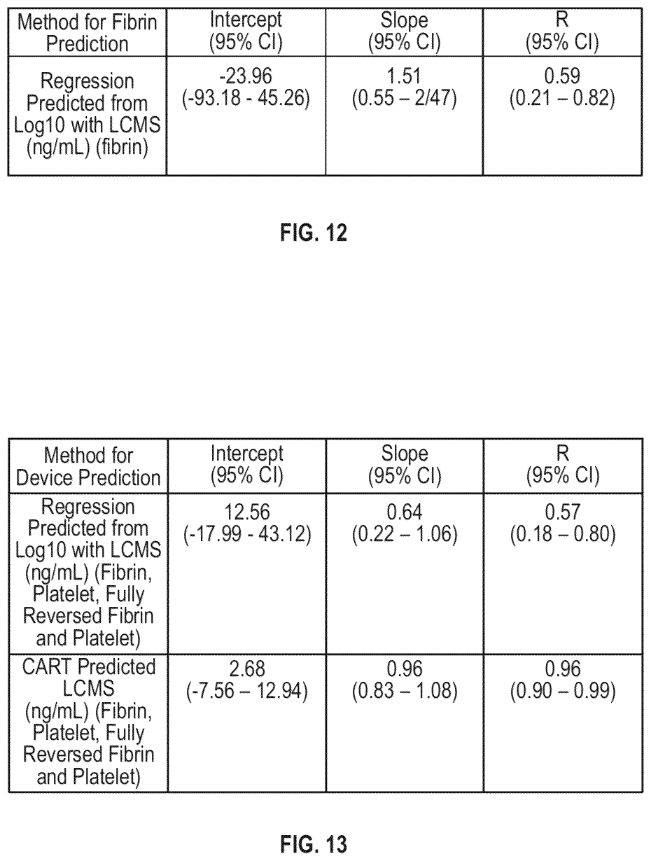

The exemplary device and system rely on analysis of both the platelet and fibrin signals, as well as the fully reversed platelet and fibrin signals, to provide an accurate quantitative determination of the DOAC. For comparison, experimentation involved a quantitative analysis that only relied on the fibrin signal to predict the DOAC concentration. FIG. 12 is a table of DOAC concentration prediction based only on the fibrin signal. FIG. 13 is a table of DOAC concentration predictions based on both fibrin and platelet signals. Both the fibrin and platelet signals were used at the time of max A along with the fully reversed fibrin and platelet signals (four input variables), and two different models were used for calculating the predictions. FIG. 13 provides the predicted DOAC concentration versus liquid chromatography with tandem mass spectrometry (LC-MS/MS).

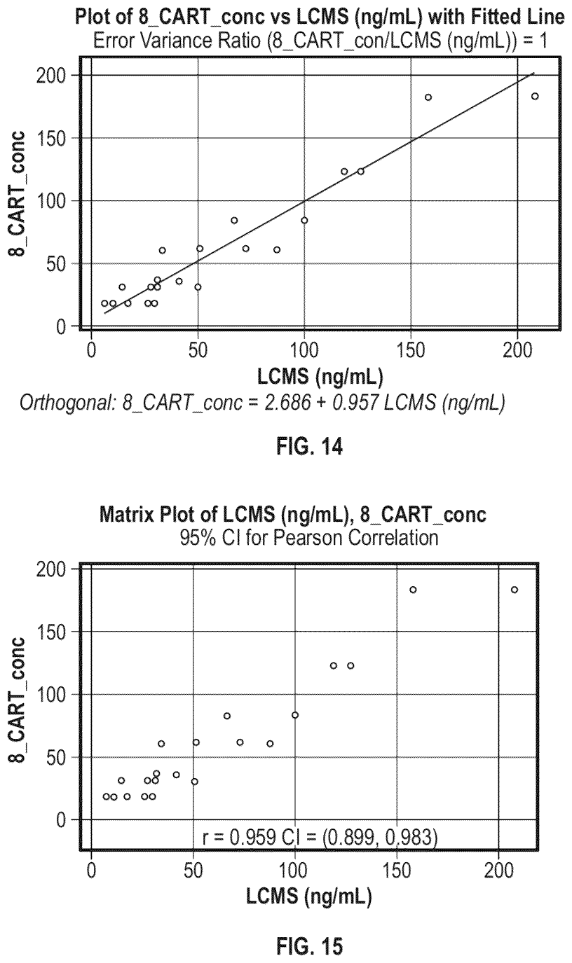

The best results were obtained from a CART Regression model. This resulted in an 8 node (7 split) decision tree to assign a concentration to each sample. FIG. 14 is a graph of DOAC concentration predictions based on both fibrin and platelet signals using the CART Regression model verses LC-MS/MS measured data. FIG. 15 is a graph of DOAC concentration predictions based on both fibrin and platelet signals using a Pearson correlation model verses LC-MS/MS measured data.

Based on the data, as a semi-quantitative method to provide discrimination around DOAC concentrations used in clinical decision making, the exemplary device testing demonstrated feasibility to achieve desirable target ROC AUC, sensitivity, specificity, PPV and NPV. Based on the data, as a quantitative method, the exemplary device testing demonstrated feasibility by using four different signals in the assay configuration and the ability to use different methods from which the concentration can be calculated (e.g., Binary Logistic Regression, CART Regression, or the like).

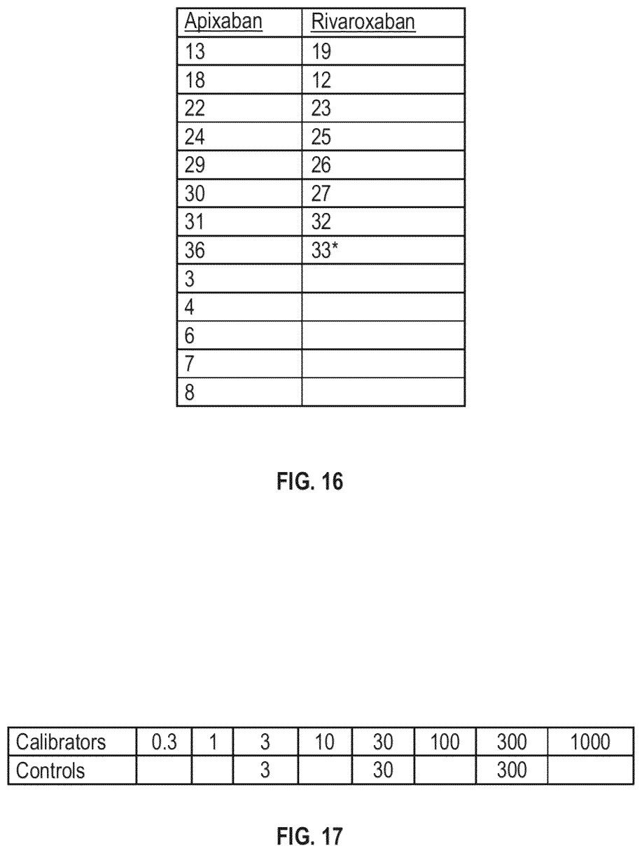

Data used in the experimentation and analysis is described below. The data included estimated DOAC concentrations (e.g., explicit concentration), and (patient sample—fully inhibited)/(full reversed—fully inhibited). In some instances, manual imaging analyses was used, which entailed modifying the region of interest to exclude aggregated material arriving on the clot zone from upstream. In an effort to determine the capability of the exemplary testing method/device without secondary, manual intervention to generate the data, the files which did not include the modified ROI were used. For the collected data, time was at max Δ (fully reversed condition—unmodified patient sample, using the fibrin signal without the platelet signal). Values for the fibrin and platelet FIs were taken from the raw data. The values of the average were used. FIG. 16 is a table of patients (based on their assigned number) for apixaban and rivaroxaban. Subject 33 did not have a value calculated for the predicted DOAC based on the time at max Δ.

Pharmaron was used to compare the results of the described exemplary system in comparison to LC-MS/MS. In the performance of LC-MS/MS calibrators and controls, an internal reference assay was performed for apixaban and rivaroxaban.

The PH LC-MS/MS assay calibration and control configurations are provided in the table of FIG. 17, also in ng/ml (provided by Pharmaron). The controls are at concentrations that are redundant with the calibrators. The calibrators and concentrations used by PH were to commercial testing standards.

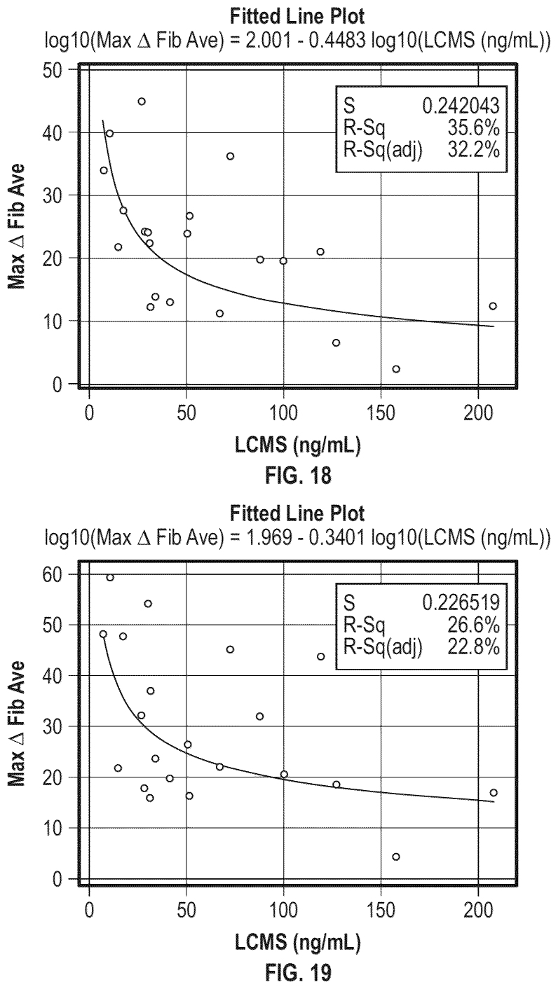

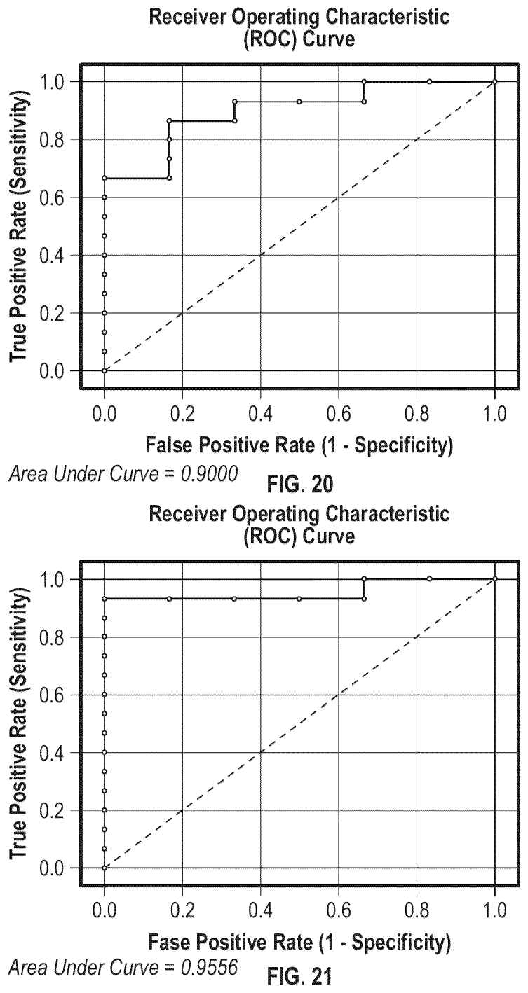

During experimentation, different time points were evaluated in terms of optimal sample analysis time. These were based on the fibrin signal. One was the time to 80 percent of maximum response, the other was the time to maximum difference between the patient sample and the 300 nM reversal reagent. All patient samples contained DOAC in this study. The relationship of the signals to the DOAC concentration was improved using the time of maximum delta (see FIGS. 18 and 19). The relationship is nonlinear. FIGS. 18 and 19 use a log10 relationship with no attempt at more refined model fitting. Specifically, FIG. 18 is a graph of a fitted line plot for a fibrin signal with a time of maximum difference between the patient sample condition and the fully reversed patient sample condition, and FIG. 19 is a graph of a fitted line plot for a platelet signal with a time of maximum difference between the patient sample condition and the fully reversed patient sample.

The exemplary system provides the potential for a multiplex, microfluidic, hemodynamic assay, which simulates physiological blood flow and measures the functional components of blood during the clotting process via fluorescent optical detection. The system can be used for in vitro diagnostic use by trained medical professionals at the point-of-care and by laboratory professionals in clinical laboratories. When used with the DOAC assay, the system provides semi-quantitative (and potentially quantitative) results for the detection of direct oral anticoagulant drugs (DOACs) in whole blood, identification of the DOAC as either Factor Xa or Direct Thrombin Inhibitor, and classification of anticoagulation relative to the concentration above or below ISTH Guidelines regarding appropriate levels to trigger reversal of anticoagulation therapy and for ivtPA thrombolysis therapy to treat a stroke. The system, in general, also provides an assessment of coagulation and platelet function in real-time.

The system can be indicated for the assessment of hemostasis status in patients who are suspected of or treated with DOACs where the evaluation of DOAC use and anti-coagulation status can aid in the assessment of bleeding and thrombotic risk and the restoration of hemostasis. The system can be used with patients in a variety of situations where DOAC evaluation could be useful to have as additional information, e.g., patients experiencing a DOAC related bleeding episode, patients at risk for major bleeding (such as those undergoing urgent invasive surgery, trauma patients, stroke patients requiring tPA), patients requiring reversal, or the like. The system could also be used to evaluate drug dosing during initial application of DOAC, as well as evaluating hemostatic response to long term DOAC use. In some instances, the assay can be used as a stand-alone test. In some embodiments, the assay can be used in conjunction with other clinical and laboratory findings.

In evaluating the relationship of the system predicted DOAC concentrations to the LC-MS/MS measured concentrations, all comparisons were made based on ng/ml because that is generally the units used for the clinical decision points regarding DOAC reversal. The data from the patient samples was evaluated for the ability to provide discrimination around the clinical decision points for DOAC reversal of 30 ng/mL and 50 ng/mL (e.g., with two decision points being used due to different clinical cutoff requirements for DOAC reversal in certain patient populations, necessitating a difference in sensitivity of the assay).

A discussion of the Binary Logistic Regression for DOAC≥30 ng/ml is provided. Using the fibrin signal at the time of max Δ to detect the DOAC concentration of ≥30 ng/ml, the receiving operating characteristic (ROC) AUC was 0.9000, with a sensitivity of 87% and a specificity of 83% (see FIG. 20). Adding the platelet signal to the model did not change the ability to detect the concentration of ≥30 ng/mL. However, adding the fully reversed fibrin signal to the model improved the discrimination with an ROC AUC of 0.9011 with a sensitivity of 80% and specificity of 100%. Using both the fibrin and platelet signals at the time of max Δ along with the fibrin and platelet signals from the fully reversed sample, the performance had an ROC AUC of 0.9556 with sensitivity of 93% and specificity of 100% (see FIG. 21).

A discussion of the Binary Logistic Regression for DOAC≥50 ng/ml is provided. The fibrin signal alone did not provide acceptable discrimination for detection of DOAC concentration≥50 ng/ml (ROC AUC 0.7182) and this was not improved by adding the platelet signal to the model. The platelet signal alone also did not demonstrate the ability to discriminate at this concentration. Using the four factor model in Binary Logistic Regression, the best performance was an ROC AUC of 0.7636. The assay discrimination at this concentration, the way the regression model operates, and the limited dataset (limited number of samples around the 50 ng/ml threshold) affected the noted results.

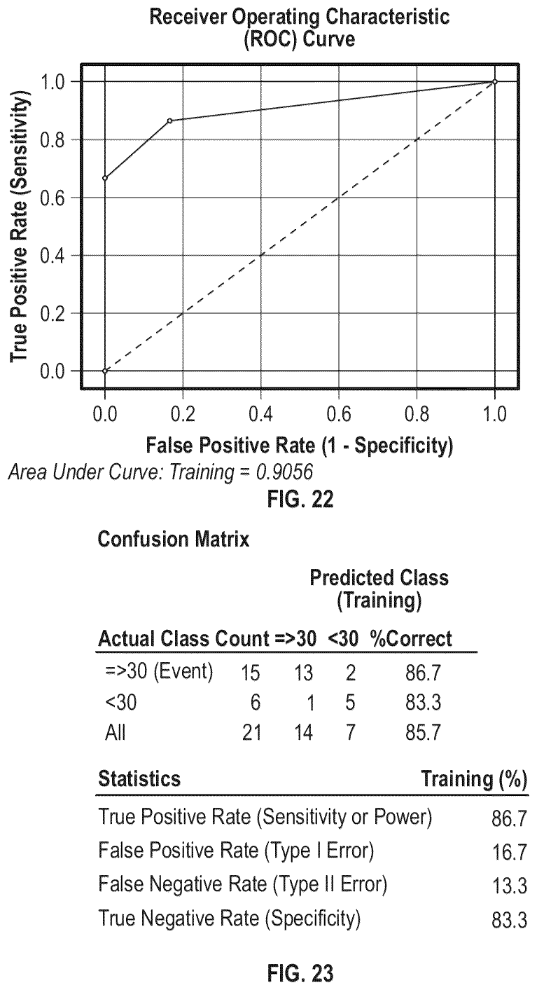

A discussion of the CART Regression for DOAC≥30 ng/mL is provided. The evaluation resulted in a 3 node classification tree with an ROC AUC of 0.9056 with sensitivity of 87% and specificity of 83% for the fibrin signal (see FIG. 22).

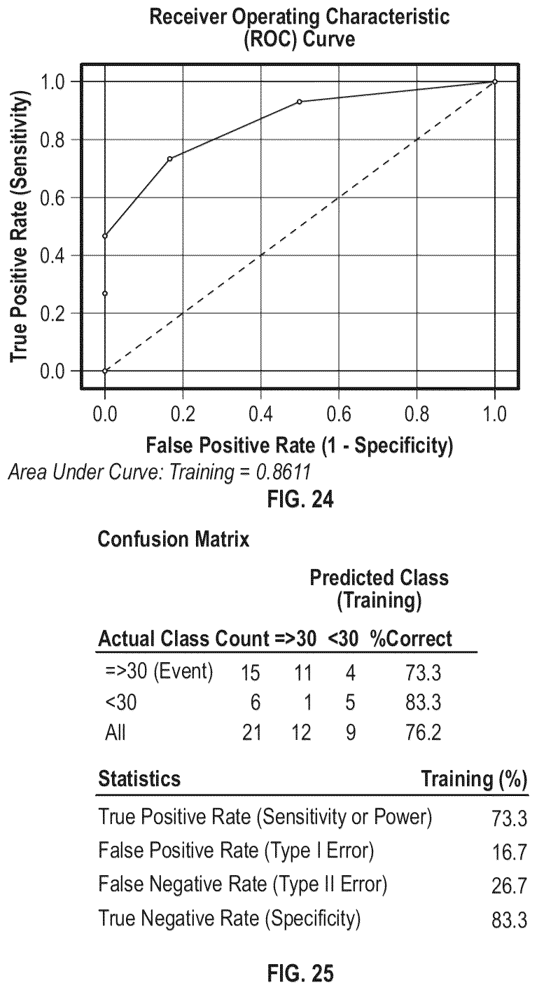

FIG. 23 is a table of a confusion matrix for only a fibrin signal, and FIG. 24 is a graph of a receiver operating characteristic (ROC) curve for only a platelet signal, using CART classification for DOAC≥30 ng/mL. The platelet signal alone resulted in a 5 node classification tree which had an ROC AUC of 0.8611 with a sensitivity of 73% and specificity of 83%. FIG. 25 is a table of a confusion matrix for only a platelet signal. This analysis was performed to evaluate if the platelet signal alone had any performance. The data showed that the platelet signal alone was providing a performance result.

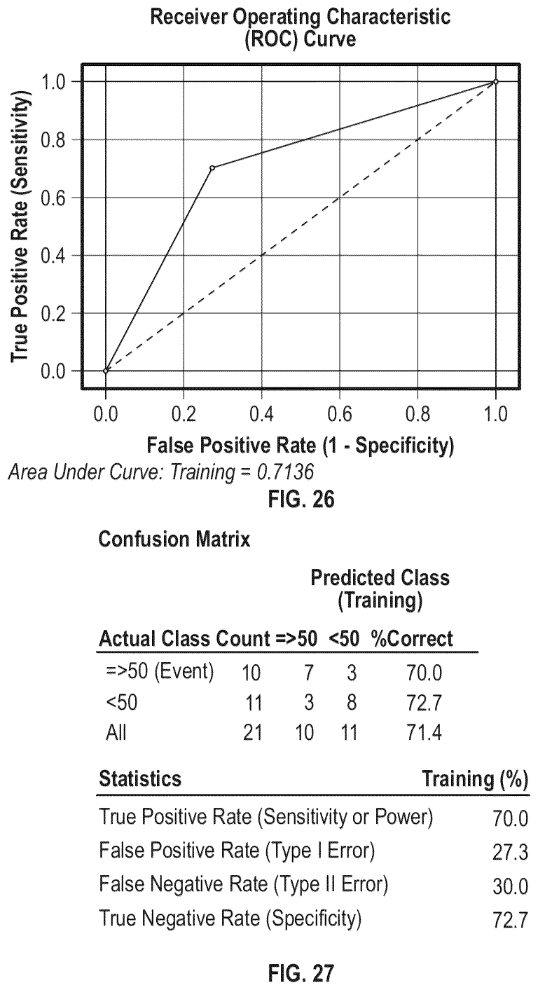

FIG. 26 is a graph of a receiver operating characteristic (ROC) curve for only a fibrin signal for DOAC≥50 ng/mL, a nd FIG. 27 is a table of a confusion matrix for only a fibrin signal. The ability of the fibrin signal alone to detect a DOAC concentration≥50 ng/ml had an ROC AUC of 0.7136, with sensitivity of 70% and specificity of 73%.

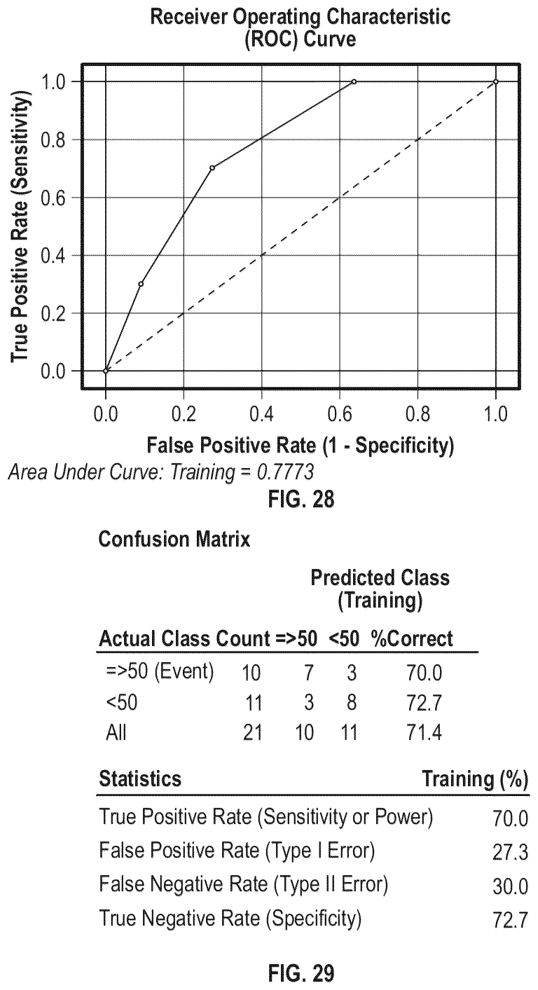

FIG. 28 is a graph of a receiver operating characteristic (ROC) curve for only a platelet signal for DOAC≥50 ng/mL, and FIG. 29 is a table of a confusion matrix for only a platelet signal. The performance of the platelet signal alone was better than fibrin alone, and resulted in a 4 terminal node decision tree with an ROAC AUC of 0.7773 with sensitivity of 70% and specificity of 73%. This analysis was performed to show that measuring the platelet signal had some performance on its own.

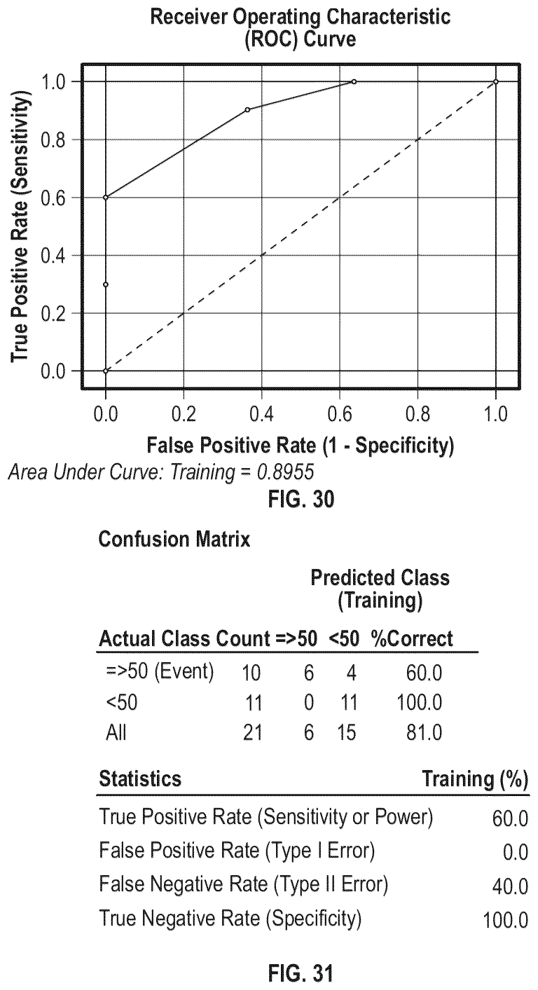

FIG. 30 is a graph of a receiver operating characteristic (ROC) curve for both a fibrin and platelet signal for DOAC≥50 ng/ml, and FIG. 31 is a table of a confusion matrix for both a fibrin and platelet signal. Using both signals at the time of maximum delta (based on fibrin) resulted in the highest ROC AUC of 0.8955 with a sensitivity of 60% and a specificity of 100%.

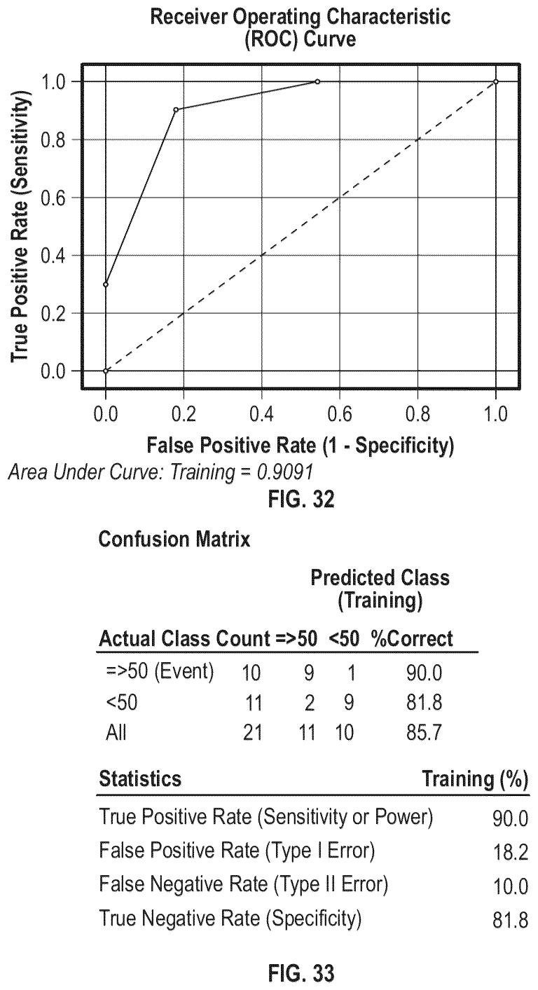

FIG. 32 is a graph of a receiver operating characteristic (ROC) curve for a fibrin and platelet signal, and for the fibrin and platelet signal from the fully reversed sample, for DOAC≥50 ng/ml, and FIG. 33 is a table of a confusion matrix for a fibrin and platelet signal, and for fibrin and platelet signal from the fully reversed sample. Using the same 4 factors that gave the best results in Binary Logistic Regression, which are the fibrin and platelet patient signal and the fibrin and platelet signals from the fully reversed sample, the ROC AUC was 0.9091 with a sensitivity of 90% and a specificity of 82%.

For comparison, the quantitative capabilities were determined based only on the fibrin signal. Results are provided using the DOAC concentration as estimated using calculations and equations based upon the fibrin signal only at the time of max A. All relationships are based upon ng/mL concentrations to facilitate interpretation relative to the clinical decision points.

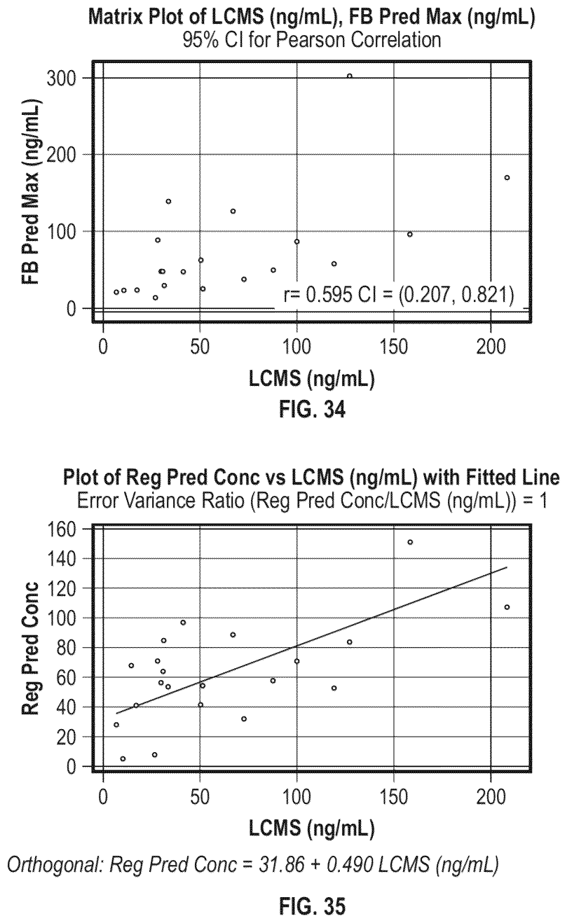

FIG. 34 is a graph of a matrix plot of LC-MS/MS and fibrin predicted concentrations, as predicted with an IC50 curve.

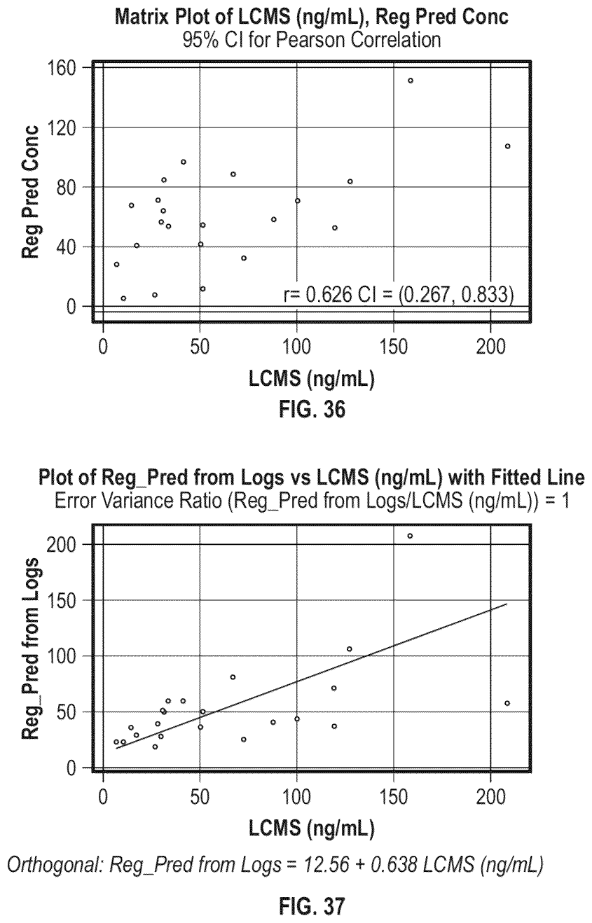

Alternative methods for quantitative capability were considered. The analyses of different models to provide for discrimination around the specific DOAC concentrations used in clinical decision making identified that the use of the unmodified sample fibrin and platelet signal, as well as the fibrin and platelet signal of the fully reversed sample at the time of max A, provided the best performance. Evaluations of new and different potential methods to estimate DOAC concentration from these signals were evaluated. One method used a regression model with the four continuous predictors versus the concentration from LC-MS/MS (without any adjustments). The results provided a regression equation with an R-sq value of 63%. However, the intercept was high and the slope was low. FIG. 35 is a graph of a regression model prediction using unmodified sample fibrin and platelet signal, as well as the fibrin and platelet signal of the fully reversed sample as compared to an LC-MS/MS measured concentration of apixaban or rivaroxaban, and FIG. 36 is a graph of a matrix plot for an LC-MS/MS prediction based on a regression model (Pearson r value correlation).

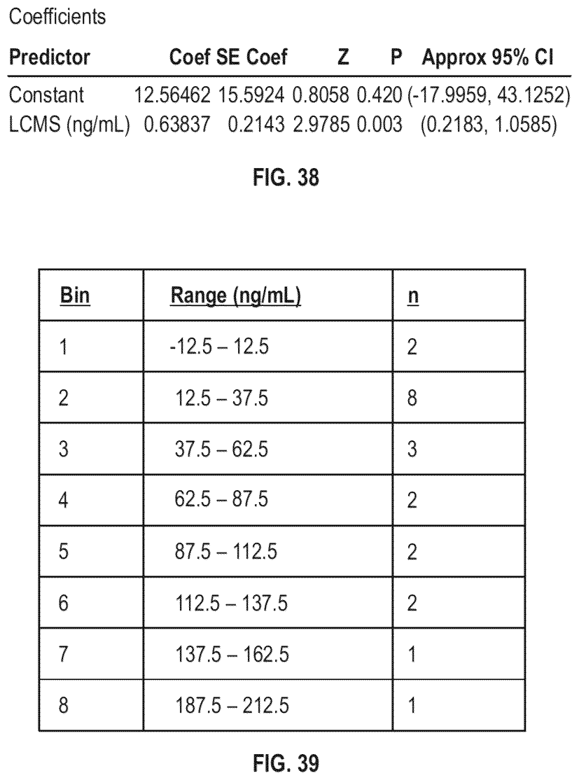

In the next iteration of the analysis, the four fibrin and platelet signals, along with the LC-MS/MS value, were converted to log10. The regression equation was again run and the fits were saved. The fits were then transformed back to ng/mL and compared to the LC-MS/MS values. While the r value is somewhat lower, the slope and intercept were improved. The precision in the range of clinical decision making was improved as well. FIG. 37 is a graph of a regression model prediction using unmodified sample fibrin and platelet signal, as well as the fibrin and platelet signal of the fully reversed sample as compared to an LC-MS/MS measured concentration of apixaban or rivaroxaban using log10, and FIG. 38 is a table of coefficients for a constant and LC-MS/MS. The data of FIG. 37 is usable for an LC-MS/MS prediction based on a regression model using log 10 to obtain the Pearson's r value. From the results, it was determined that it would be possible to use the exemplary device testing methodology output from the four signals of interest to generate a predicted value for concentration using a standard type of regression analysis. Of particular interest from this analysis was the apparent precision in the concentration ranges in which clinical decisions are made.

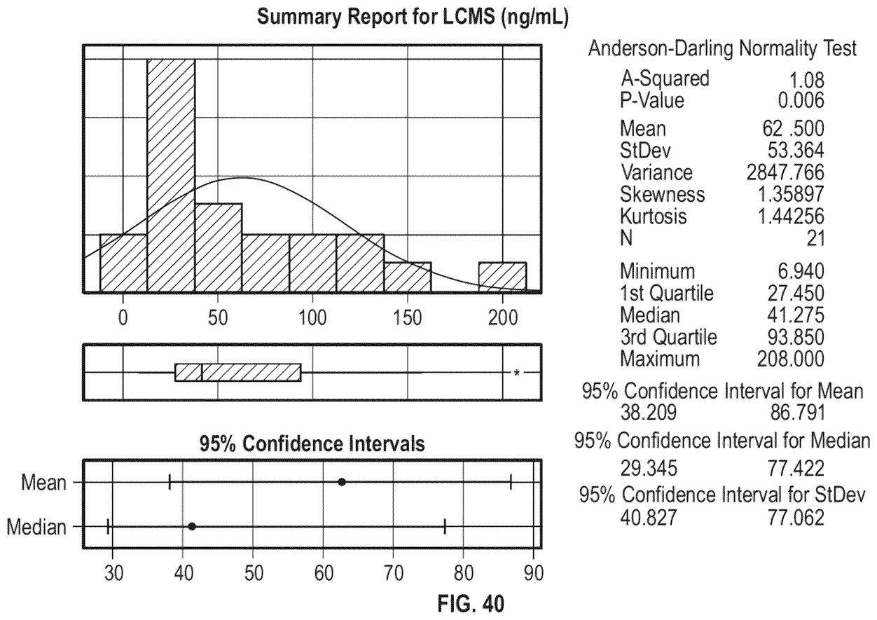

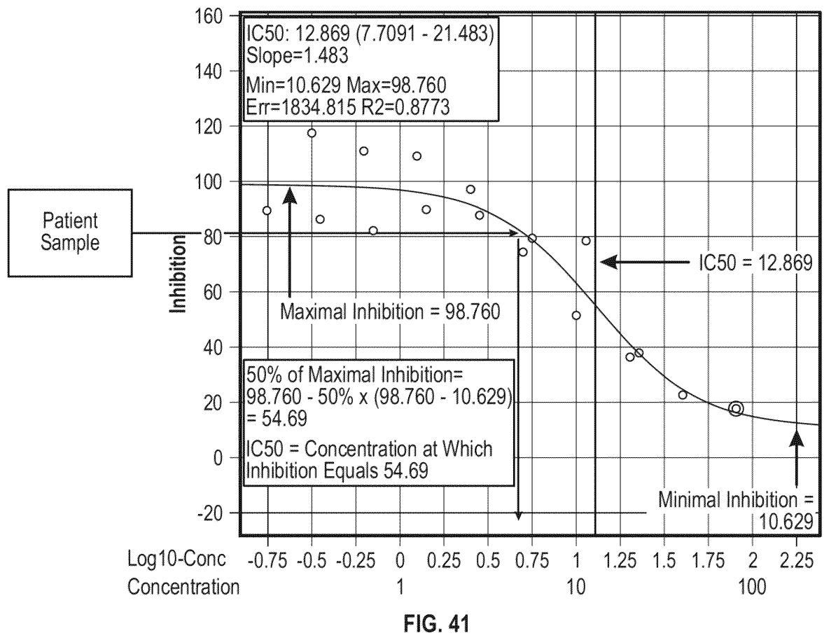

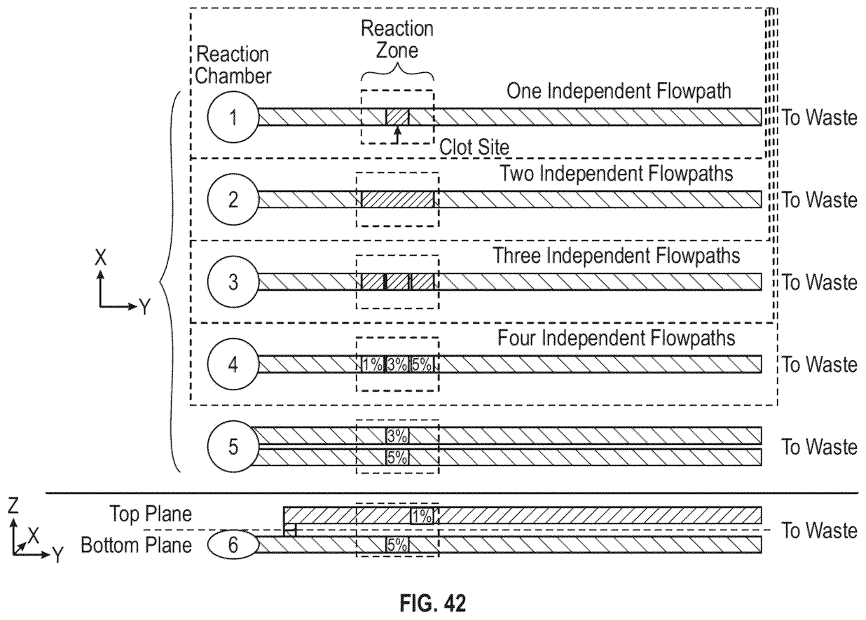

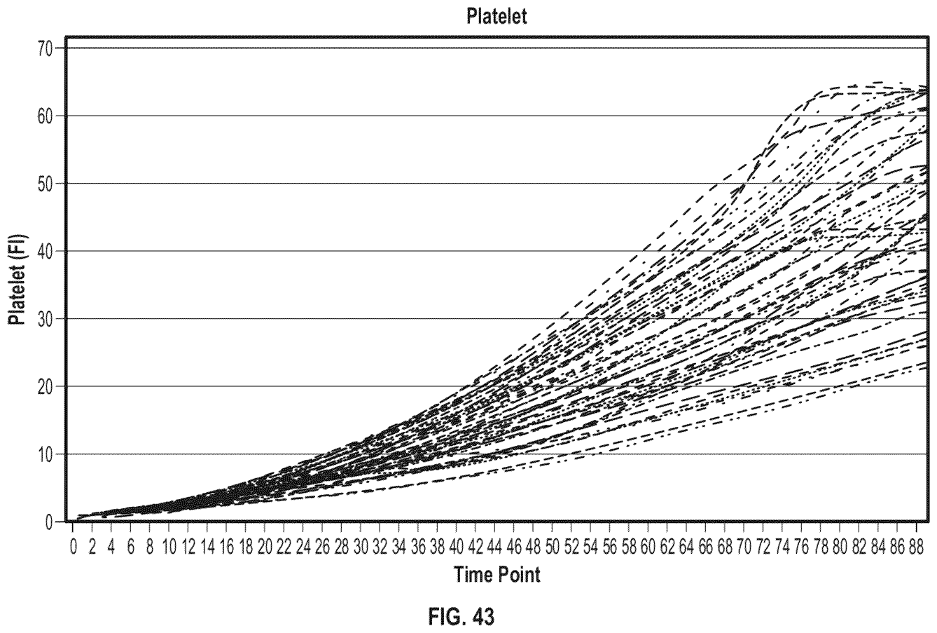

As an alternative to the standard regression model described above, CART Regression was also used for the same set of four signals to predict drug concentration from LC-MS/MS. By examining the distribution of the LC-MS/MS results, it was determined that values were not normally distributed (A-sq p value=0.006), and instead skewed to the right (FIG. 40). The histogram clusters the results into 8 bins. FIG. 39 is a table of bins and LC-MS/MS measured apixaban or rivaroxaban concentration from the distribution of the LC-MS/MS results, and FIG. 40 is a histogram of LC-MS/MS measured apixaban or rivaroxaban concentration from the distribution of the LC-MS/MS results.

For the CART Regression, the options selected used 6 samples to split a node, and 2 to be in a final node (analogous to a bin). This resulted in an 8 node (7 splits) decision tree to assign a concentration to each sample. This matched the distribution of the actual LC-MS/MS results. FIGS. 14 and 15 illustrate the results of using Orthogonal Regression and Pearson correlation versus the LC-MS/MS measured values. In particular, FIG. 14 is a graph of a CART regression model prediction using unmodified sample fibrin and platelet signal, as well as the fibrin and platelet signal of the fully reversed sample as compared to an LC-MS/MS measured concentration of apixaban or rivaroxaban, and FIG. 15 is a graph of a matrix plot for an LC-MS/MS prediction based on a CART regression model. The results demonstrate that the CART algorithm using the four signals from the exemplary device testing method can result in a determination of DOAC concentration in close agreement with that determined using LC-MS/MS.

While exemplary embodiments have been described herein, it is expressly noted that these embodiments should not be construed as limiting, but rather that additions and modifications to what is expressly described herein also are included within the scope of the invention. Moreover, it is to be understood that the features of the various embodiments described herein are not mutually exclusive and can exist in various combinations and permutations, even if such combinations or permutations are not made express herein, without departing from the spirit and scope of the invention.

1. A system for detecting and quantifying drug and/or chemical interactions with a biological sample, the system comprising:
a detection instrument with computing capability; and
an assay device capable of receiving a biological sample, wherein introduction of the biological sample into the assay device results in a biological process by which fibrin and platelets may accumulate at a reaction zone of the assay device;
wherein the assay device is capable of receiving one or more chemical reagents compatible with the biological sample and usable for detecting the accumulation of the fibrin and platelets within the reaction zone;
wherein the assay device is capable of receiving one or more drug reagents compatible with the biological sample and usable for modifying the accumulation of the fibrin and platelets within the reaction zone; and
wherein the fibrin and platelets, and their associated signals, accumulated at the reaction zone of the assay device are usable to determine at least one of a drug presence, a drug class, a drug level in relation to a threshold, or a drug concentration, within the biological sample.

2. The system of claim 1, comprising a fluorescent assembly capable of detection of the biological process by way of fluorescent labeling and detection of a resulting accumulating fluorescent signal, and comprising a processing device configured to receive as input a measurement of the fibrin and platelets to determine at least one of the drug presence, the drug class, the drug level in relation to the threshold, or the drug concentration, within the biological sample, and the processing device is configured to correlate a measured fluorescent intensity with the accumulation of the fibrin and platelets in microfluidic flow paths of the assay device.

3. The system of claim 1, wherein the one or more chemical reagents or the one or more drug reagents is a fluorescent reagent capable of labeling the fibrin and platelets from the biological sample that results in a fluorescent assembly that reports the accumulation of the fibrin and platelets.

4. The system of claim 1, comprising a light source for monitoring clot development in microfluidic flow paths of the assay device and for detecting a fluorescent reaction of the one or more chemical reagents or the one or more drug reagents.

5. The system of claim 1, wherein the assay device includes:
a first inlet port, a second inlet port, a third inlet port, and a fourth inlet port, each configured to receive the biological sample, wherein the biological sample is an unmodified sample including a platelet specific label and a fibrin specific label;
an outlet port; and
microfluidic flow paths fluidically connecting each of the first, second, third and fourth inlet ports with the outlet port;
wherein the introduction of the biological sample into the respective first, second, third and fourth inlet ports generates a fibrin signal and a platelet signal.

6. The system of claim 5, wherein at least one of the first, second, third and fourth inlet ports is configured to receive a molar excess of drug to provide a fully attenuated fibrin or platelet signal.

7. The system of claim 5, wherein at least one of the first, second, third and fourth inlet ports is configured to receive a first reversal drug to identify a first class of drug present in the biological sample, and wherein at least one of the first, second, third and fourth inlet ports is configured to receive a second reversal drug to identify a second class of drug present in the biological sample.

8. The system of claim 7, wherein the drug is a direct-acting oral anticoagulant (DOAC), and wherein the first and/or second reversal drug inhibits, antagonizes or attenuates the activity of a Xai or DTi class DOAC.

9. The system of claim 8, wherein the drug is an anti-platelet medication, and wherein the first and/or second reversal drug inhibits, antagonizes or attenuates an activity of the anti-platelet medication.

10. The system of claim 8, comprising a processing device configured to manipulate the biological sample and monitor a direct response of the biological sample to at least one of (i) the first and/or second reversal drug to identify the drug class present in the biological sample, and (ii) a molar excess of the fibrin or platelet attenuating drug to identify a drug or chemical level or concentration present in the biological sample.

11. The system of claim 5, comprising a processing device configured to compare the fibrin or platelet signal to the fully recovered fibrin or platelet signal, compare the fibrin or platelet signal to the fully attenuated fibrin or platelet signal, and compare all other coincident signals for all reactions to determine the drug presence, the drug class, the drug level in relation to the threshold, or the drug concentration, in the biological sample.

12. The system of claim 1, wherein the reaction zone comprises at least one of (i) a single flow path with separate clot sites in a serial configuration having different tissue factor (TF) concentrations, and (ii) two flow paths in parallel alone the same plane, each of the flow paths having different tissue factor (TF) concentrations.

13. The system of claim 1, wherein the reaction zone comprises two flow paths on separate planes of the assay device, the two flow paths each having a clot site in a non-overlapping configuration relative to each other and having different tissue factor (TF) concentrations.

14. A method for drug or chemical detection and quantification, the method comprising:
adding a biological sample to an assay device;
adding one or more chemical reagents to the assay device to generate a biological process by which fibrin and platelets may accumulate at a reaction zone of the assay device;
detecting the biological process with a fluorescent assembly by way of fluorescent labeling and detection of a resulting accumulating fluorescent signal; and
using the fibrin and platelets accumulated at the reaction zone of the assay device, and the accumulating fluorescent signal, to determine at least one of a drug presence, a drug class, a drug level in relation to a threshold, or a drug concentration.

15. The method of claim 14, comprising:
adding the biological sample to a first inlet port, a second inlet port, a third inlet port, and a fourth inlet port of the assay device, the assay device including an outlet port and microfluidic flow paths fluidly connecting each of the first, second, third and fourth inlet ports with the outlet port, wherein the biological sample is an unmodified sample including a platelet specific label and a fibrin specific label;
generating a fibrin signal and a platelet signal from the biological sample for each of the first, second, third and fourth inlet ports; and
determining the drug presence, the drug class, the drug level in relation to the threshold, or the drug concentration from the fibrin signal and the platelet signal.

16. The method of claim 15, wherein the biological sample comprises a raw blood sample, a processed blood sample, a blood sample treated with an anticoagulant to prevent intrinsic pathway coagulation activation, a citrated blood sample that is recalcified, or a blood sample treated with an antiplatelet drug to prevent platelet activation.

17. The method of claim 15, comprising identifying an overall state of coagulation from the unmodified sample by evaluating the fibrin signal and a fully recovered fibrin signal obtained using a reversal agent.

18. The method of claim 15, comprising identifying an overall state of platelet function from the unmodified sample by evaluating the platelet signal and a fully recovered platelet signal obtained using a reversal agent.

19. The method of claim 15, comprising using a Xai reversal agent in the second inlet port and a DTi reversal agent in the third inlet port.

20. The method of claim 15, comprising receiving a concentration of a fibrin attenuating drug to a point which produces no further attenuation of the fibrin signal to obtain a fully attenuated fibrin signal.

21. The method of claim 15, comprising receiving a concentration of a platelet attenuating drug to a point which produces no further attenuation of the platelet signal to obtain a fully attenuated platelet signal.

22. The method of claim 15, comprising generating a platelet signal coincident with the fibrin signal for each of the first, second, third and fourth inlet ports.

23. The method of claim 15, wherein an unmodified sample fibrin and an unmodified sample platelet signal are generated from a microfluidic flow path associated with the first inlet port, a fully reversed fibrin signal and a coincident platelet signal are generated from either a microfluidic flow path associated with the second or third inlet port by way of interaction with a Xai or DTi reversal agent, and a fully attenuated fibrin signal and a coincident platelet signal are generated from a microfluidic flow path associated with the fourth inlet port.

24. The method of claim 15, comprising imparting a light source onto the microfluidic flow paths to monitor clot development in the microfluidic flow paths based on a fluorescent reaction of the one or more reagents, receiving as input at a processing device a measured fluorescent intensity of the monitored clot development, and correlating with the processing device the measured fluorescent intensity with platelet and fibrin accumulation in the microfluidic flow paths.

25. The method of claim 15, comprising comparing with a processing device the fibrin signal to a fully reversed fibrin signal, and a fully inhibited fibrin signal along with coincident platelet signals, to determine the drug class or drug concentration in the biological sample.
